(19) United States
(12) Patent Application Publication
(10) Pub. No.: US 2025/0098665 A1
(43) Pub. Date: Mar. 27, 2025

(54) MAIZE POLLEN STORAGE AND CARRIERS

(71) Applicant: Syngenta Crop Protection AG, Basel (CH)

(72) Inventors: Jared CARTER, Research Trianlge Park, NC (US); Jay Austin DINWIDDIE, Research Triangle Park, NC (US); Nicholas D. Polge, Vero Beach, FL (US)

(73) Assignee: Syngenta Crop Protection AG, Basel (CH)

(21) Appl. No.: 18/976,437

(22) Filed: Dec. 11, 2024

Related U.S. Application Data
(62) Division of application No. 18/861,930, filed on Jan. 1, 0001.
(60) Provisional application No. 63/346,066, filed on May 26, 2022.

(51) Int. Cl. A01N 3/00; A01H 1/02; A01H 6/46

(52) U.S. Cl. CPC A01N 3/00; A01H 1/02; A01H 6/4684

(57) ABSTRACT

Maize pollen is notoriously fragile and susceptible to degradation unless adequately stored. Unlike some tree pollen, which can be quite hardy and capable of successful fertilization for months or years after it is shed, maize pollen remains viable for mere hours after shedding before it begins to degrade. Described here is an invention for storing maize pollen where the pollen is collected and stored in a refrigerated, but not frozen, environment with a breathable barrier. Pollen stored as described herein may remain viable for up to twelve days, or two weeks, or longer. Adding a carrier compound can extend viability of the pollen.

### FIELD OF THE INVENTION

This invention relates to the field of maize breeding and human-induced pollination, and particularly the field of collecting, storing, and applying stored maize pollen in maize production fields and greenhouses.

### BACKGROUND

Pollen storage has long been both a need and a goal for plant breeders. See generally W. M. King, Report of chief on seed divisions, In REPORT OF THE COMMISSIONER OF AGRICULTURE (YEARBOOK), Washington D.C., GPO, 47-61 (1885) (articulating the desire for stored pollen “so that we might use it when and where convenient to ourselves.”). In some plants, pollen is quite hardy and long-lived. For example, gingko tree pollen can be collected and stored for six months or more with no specific care required. In contrast, other plants have pollen that is fragile and susceptible to rapid decay within hours if left exposed to the elements. Maize (corn) is one such plant.

In maize commercial hybrid production fields, current practice is to alternate four rows of female inbred plants with two rows of male inbred plants. The females are detasseled to prevent self-pollination, while the males are grown solely for their ability to pollinate the neighboring females. This arrangement works best where the female plants and the male plants are of similar maturity groups that is, the males shed pollen at about the same time the females are receptive to the pollen.

However, a risk with current practices is a possibility of unsuccessful pollination, and therefore the loss of a crop, if the males and the females are of different maturity groups. Without pollen storage, the grower risks having the male plant shed pollen too early or too late and could lose an entire field due to failed pollinations. With pollen storage, pollen could be delivered at precisely the right time regardless of flowering time challenges. Interbreeding different maturity groups could be more easily accomplished, thus expanding the genetic pool and improving maize plant breeding, for example, by making maize lines that are more drought and/or disease resistant.

### SUMMARY

Growers need an ability to reliably collect and store maize pollen on one day or in one location and deliver that pollen to a field of females another day or at another location. To meet this need, a method of storing maize pollen is provided. In one embodiment, one collects an amount of fresh maize pollen; optionally treats the collected pollen with a carrier; seals the pollen in a container with a breathable barrier and stores the pollen in a refrigerated environment. Pollen collected and stored in this manner remains viable for up to 20 days, and at least up to 12 days. In one aspect, the carrier is talc powder, or silica powder. In another aspect, the carrier is a metallic powder or mica mineral. The carrier may be applied in a pollen:carrier ratio of 1:2; 1:1, 2:1, 3:1, 4:1, 5:1, 6:1, 7:1, 8:1, 9:1, 10:1, 20:1, 30:1, 40:1, 50:1, and any ratio between 1:2 and 50:1. Preferably, the pollen:carrier ratio is 2:1. In one aspect, the breathable barrier may be Parafilm, Tyvek, 3M Micropore tape, cellulose, nitrocellulose, and a non-airtight container. Preferably, the breathable barrier is 3M micropore tape. In another aspect, a carbon dioxide sequestering agent may be added to the sealable container. Additionally, provided herein is a pollen storage vessel with a total breathable barrier surface area. The pollen storage vessel may have multiple openings and said openings may be covered in a breathable barrier.

### BRIEF DESCRIPTION OF THE FIGURES

FIG. 1 is a photo of the seed set produced using pollen stored in a breathable barrier surface area factorial using Tyvek and Micropore breathable barriers covering perforations in otherwise airtight storage containers with added soda lime (Example 4, Experiment 5). From left to right, the ears were produced using pollen stored in an otherwise airtight container with a single 1.588 mm (⅙-inch) opening covered in Tyvek, a single 3.175 mm (⅛-inch) opening covered in Tyvek, a single 9.525 mm (⅜-inch) opening covered in Tyvek, a single 1.588 mm ( 1/16-inch) opening covered in Micropore tape, a single 3.175 mm (⅛-inch) opening covered in Micropore tape, a single 6.35 mm (¼-inch) opening covered in Micropore tape, and a single 9.525 mm (⅜-inch) opening covered in Micropore tape treatments, respectively.

FIG. 2 is a photo of the seed set produced using pollen stored in a breathable barrier surface area factorial using open perforations in otherwise airtight storage containers with added soda lime (Example 4, Experiment 6). From left to right, the ears were produced using pollen stored in an otherwise airtight container with a single 3.175 mm (⅛-inch) open perforation covered in Tyvek, a single 1.588 mm ( 1/16-inch) open perforation, a single 3.175 mm (⅛-inch) open perforation, a single 6.35 mm (¼-inch) open perforation treatments, respectively, and the final two ears on the right are from an otherwise airtight container with a single 9.525 mm (⅜-inch) open perforation.

FIG. 3 is a photo of the seed set produced using pollen stored in a breathable barrier surface area factorial using Tyvek and Micropore breathable barriers covering large perforations in otherwise airtight storage containers with added soda lime (Example 4, Experiment 7). The three ears farthest to the left in the image are from treatments where large perforations were covered in Micropore tape. Perforation size increases from left to right across the three ears. The three ears farthest to the right in the image are from treatments where large perforations were covered in Tyvek. Perforation size increases from left to right across the three ears.

FIG. 4 is a photo showing the seed set produced using pollen stacked 6.5 cm deep in a 50 ml conical tube with a 71.3 mm² opening covered in micropore tape with no soda lime added. (Example 4, Experiment 8). The left most ear is from the top 1.5 cm portion of the pollen carrier mix stacked in the tube. The middle ear is from the central portion of the pollen stack approximately 4 cm below the surface. The right most ear is from the base portion of the pollen stack 6.5 cm from the surface.

FIG. 5 is a photo of the seed set produced using pollen stored in a breathable barrier surface area using Micropore breathable barriers covering perforations in otherwise airtight storage containers with and without added soda lime (Example 4, Experiment 9). The four ears on the left side of the images are from treatments where no soda lime was added. The four ears on the right side of the image are from treatments with added soda lime. For each series of four ears, perforation size increases from left to right.

FIG. 6 shows pressure normalized percent oxygen during 9-day pollen storage from Example 6.

FIGS. 7-12 show the stock version of the culture flask.

FIG. 7 shows a VWR® Cell Culture Flask with the stock vented cap removed and 3M micropore tape (shown using the black arrow) used as a breathable barrier. This figure shows the larger surface area flat side that served to be the upward facing top of the flask during use.

FIG. 8 shows a VWR® Cell Culture Flask with the stock vented cap removed and 3M micropore tape (shown using the black arrow) used as a breathable barrier. This figure shows the smaller surface area flat side of the flask that served as the downward facing bottom side of the flask during use. It is lower in surface area due to a taper manufactured into the 47 mm exterior height sidewalls of the flask.

FIG. 9 shows a VWR® Cell Culture Flask with the stock vented cap removed and 3M micropore tape used as a breathable barrier. This figure shows a side view of the flask as it stands up on the end opposite the end of the 3M micropore tape breathable barrier.

FIG. 10 shows a VWR® Cell Culture Flask with the stock vented cap removed and 3M micropore tape (shown using the black arrow) used as a breathable barrier. This figure shows an angled top and side view as the flask sits on the downward facing bottom with the tapered sidewalls.

FIG. 11 shows a VWR® Cell Culture Flask with the stock vented cap removed and 3M micropore tape (shown using the black arrow) used as a breathable barrier. This figure shows the flask from the end opposite the vessel the 3M micropore tape breathable barrier with the flask sitting downward facing.

FIG. 12 shows a VWR® Cell Culture Flask with the stock vented cap removed and 3M micropore tape (shown using the black arrow) used as a breathable barrier. This figure shows a side view of the flask as it stands up on the end opposite the 3M micropore tape breathable barrier. This is the opposite side of the view in FIG. 9.

FIGS. 13-20 portray a 22-hole configuration of the pollen storage vessel.

FIGS. 13-14 show a VWR® Cell Culture Flask with 11 holes drilled into the larger surface area flat side facing up (FIG. 13) and 11 holes drilled into the smaller surface area side with tapered sidewalls (FIG. 14). The 22 total holes are indicated by black arrows—11 per figure.

FIGS. 15-16 show a VWR® Cell Culture Flask with 11 holes drilled into the larger surface area flat side facing up. FIG. 15 is a view from the side while FIG. 16 is a view from the end opposite the vent cap. The 11 holes drilled into the larger surface area flat side facing up are indicated by black arrows.

FIGS. 17-20 show a VWR® Cell Culture Flask with 11 holes drilled into the larger surface area flat side facing up and 11 holes drilled into the smaller surface area side with tapered sidewalls. However, the 22 total holes are covered by 3M micropore tape that serves as the breathable barrier. FIGS. 17-19 are views with the larger surface area flat side facing upwards; FIG. 17 shows the cap side closest to the camera while FIG. 18 is from the end opposite of the stock cap. FIG. 19 is a side view with stock cap on the left side. FIG. 20 is a view of the smaller surface area side with tapered sidewalls and faces downward during use. A representation of the 3M micropore tape is indicated by black arrows.

FIGS. 21-33 portray a 28-hole configuration of the pollen storage vessel.

FIGS. 21-23 show a VWR® Cell Culture Flask with 11 holes drilled into the larger surface area flat side facing up and 11 holes drilled into the smaller surface area side with tapered sidewalls. FIG. 21 is viewed with the stock cap closest to the camera, FIG. 22 is viewed from the end opposite the stock cap, and FIG. 23 is a side view with the stock cap to the left.

FIG. 24 shows a VWR® Cell Culture Flask from the side while standing up right on the end opposite the stock cap (vertically). This view shows 3 holes drilled into the side of the flask.

FIG. 25 shows a VWR® Cell Culture Flask with the smaller surface area side with tapered sidewalls facing up. The 11 holes are drilled into the flat side.

FIG. 26 shows a VWR® Cell Culture Flask from the side while standing up right on the end opposite the stock cap (vertically). This view shows 3 holes drilled into the side of the flask.

FIG. 26 is the opposite side shown in FIG. 24.

FIGS. 27-33 show the 28-hole configuration culture flask but the holes are covered with 3M micropore tape as a breathable barrier. A representation of the micropore tape is indicated by black arrows.

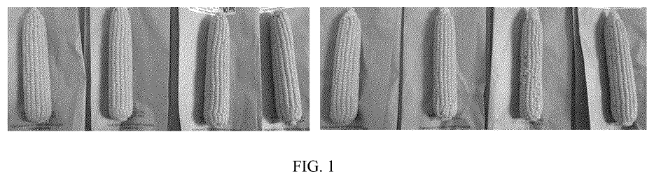

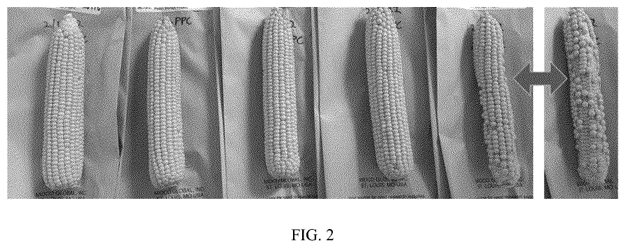

### DEFINITIONS

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, a “container” or “vessel” refers to an object capable of holding pollen within it. For example, a container may refer to a Magenta GA7 box. Said container or vessel also comprises a breathable barrier. As used herein, “Breathable Barrier” refers to a component of a storage container or vessel for maize pollen. The breathable barrier component of the container or vessel allows for sufficient gas exchange with minimal water vapor transmission. Breathable barriers may include, but are not limited to, Parafilm, Tyvek, Micropore tape, perforations in an otherwise airtight storage container or vessel (e.g., mason jar and lid), or use of non-airtight containers or vessels with aperture for gas exchange manufactured to a specified total surface area (e.g., clamshell container, Magenta GA7 box, VWR® cell culture flasks 25-850 ml capacity). For cell culture flasks, see generally us.vwr.com/store/product/12585790/vwr-cell-culture-flasks. A non-airtight container may also include, for example, a mason jar and with a perforated lid.

“Carbon dioxide sequestration,” as used herein means carbon dioxide (“CO₂”) is captured by way of a carbon dioxide sequestering agent, e.g., soda lime, activated carbon, ethanolamine, Zeolite 4A, lithium hydroxide (LiOH), or activated magnesium silicate (e.g., FLORISIL®). In this manner, excessive CO₂ buildup in a chamber is prevented. Optionally, a sequestration agent will prevent CO₂ from exceeding 10 mmol CO₂ per liter of chamber headspace.

“Carrier,” as used herein, means a compound, preferably in powdered form, which acts as an agent to accompany collected pollen. Suitable carrier compounds can be, but are not limited to, talc powder, silica powder, and the like.

“Clumping,” “Aggregating,” and similar terms, as used herein, refers to the tendency of pollen to bind together, whether due to excess moisture or other cause, in the absence of a carrier and/or suitable storage conditions. Pollen that has clumped is not flowable and cannot be blown by air onto a silk. Clumped pollen is unlikely to adhere to a silk sufficiently to cause pollination.

As used herein, the term “comprising” or “comprise” is open-ended. When used in connection with a method comprising a series of steps, that method is still practiced so long as the series of steps are performed, even if additional steps are performed.

“Crystalline silica,” as used herein, refers to a powdered form of silica derived from quartz or other natural rock formations. The terms “crystalline silica,” “SiO₂,” and “polycrystalline silica” are used interchangeably throughout. Crystalline silica has different structural properties than talc or amorphous silicas, which include but are not limited to a higher Mohs mineral hardness, higher bulk density, and lower specific surface area. In one embodiment, the crystalline silica comprises an average particle size between 1 nanometer (1 nm) and 100 micrometers (100 μm). In another embodiment, the crystalline silica comprises an average particle size between 1 micrometer (1 μm) and 10 micrometers (10 μm). Unless otherwise specified, particle size values provided herein are the average size.

“Activated magnesium silicate” as used herein, refers to a synthetic powdered magnesium silicate. The terms “activated magnesium silicate,” “synthetic amorphous activated magnesium silicate,” and “MgO₃Si” are used interchangeably throughout. “FLORISIL®” is a commercially available source of activated magnesium silicate. See www.ussilica.com/products/florisil. Activated magnesium silicate is characterized by an amorphous structure and high specific surface area. In one embodiment, the activated magnesium silicate comprises an average particle size between 75 micrometers (75 μm) and 149 micrometers (149 μm). In another embodiment, the activated magnesium silicate comprises an average particle size of less than 75 micrometers (<75 μm).

As used herein, the term transgenic “event” refers to a recombinant plant produced by transformation and regeneration of a single plant cell with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. Normally, transformation of plant tissue produces multiple events, each of which represent insertion of a DNA construct into a different location in the genome of a plant cell. Based on the expression of the transgene or other desirable characteristics, a particular event is selected. Thus, for example, “event 3272”, “3272” or “3272 event” as used herein, means the original 3272 transformant and/or progeny of the 3272 transformant and/or plants derived in any way from the original 3272 transformant. For 3272, See WO06/098952.

Other examples of transgenic events include, but are not limited to, MIR162 (See WO07142840), Bt11 (See U.S. Pat. No. 6,114,608 (construct) and WO8705629 (gene)), GA21 (See WO9704103 (gene) WO9844140 (cassette)), MIR604 (See WO05103301), MZIR098 (See WO18231890), 5307 (See WO10077816), DAS40278 (See U.S. Pat. No. 8,598,413), TC1507 (See WO04099447), DAS-59122-7 (See WO06/039376), NK603 (See U.S. Pat. No. 6,825,400), MON810 (See U.S. Pat. No. 6,713,259), MON863 (See U.S. Pat. No. 7,705,216), MON89034 (See WO07140256), MON88017 (See WO05059103), DP-4114 (See WO11084621), and MON87411 (See WO13169923).

“Flowable,” as used herein, means the ability of a powder-like substance to be easily moved by application of air, wind, sound, or to be poured with unbroken continuity and proceed steadily and easily.

As used herein, the term “gas” refers to a gas mixture (e.g., the normal air composition or a gaseous combination of oxygen-enriched air) or a substantially pure gas (e.g., pure oxygen). In one embodiment, when referring to gas, one may be referring to oxygen and carbon dioxide.

“Heterotic group,” as used herein, refers to a breeding categorization of inbred line.

“IHeterotic group” and “heterotic pool” are used interchangeably and refer to the relationship between breeding pools of maize populations. Broadly, the primary designations for heterotic pool are: Stiff Stalk (“SS,” also called Iowa Stiff Stalk Synthetic, or “BSSS”), Non Stiff Stalk (“NSS”), and Iodent (“IDT”). See J. v. Hweerwaarden, et al., Historical genomics of North American maize, PROC. NAT'L ACAD. SCI. U.S.A. 109(31): 12420-25 (2012). These are not exclusive, however, and other designations are known, e.g., Lancaster Sure Crop (“LSC”). See, e.g., C. Livini, et al., Genetic diversity of maize inbred lines with and among heterotic groups revealed by RFLPs, THEOR. APPL. GENET. 84: 17-25 (1992). See further Hallauer et al. (1998) CORN BREEDING, p. 463-564; G. F. Sprague and J. W. Dudley (ed.) CORN AND CORN IMPROVEMENT; Smith, et al. (1990) Theor. Appl. Gen. 80:833-840; Mikel and Dudley (2006) Crop Set 46: 1193-1205. See also WO2020/205334 and WO2021/041077, incorporated herein by reference in their entireties.

The term “germplasm” refers to the totality of the genotypes of a population or other group of individuals (e.g., a species or plant line). The phrase “adapted germplasm” refers to plant materials of proven genetic superiority; e.g., for a given environment or geographical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, the term “mica” refers to the general chemical formula X2Y4-6Z8O20(OH, F)4, in which X is an alkali metal or alkaline earth metal, Y is a transition metal, post-transition metal, or alkaline earth metal, and Z is silicon, aluminum, or may include other transition metals.

“Starting oxygen content,” as used herein, refers to the amount of oxygen present (whether measured as an absolute measurement, a percentage, or otherwise) in the atmosphere of a chamber comprising collected pollen at its outset and once initially sealed. In one embodiment, the starting oxygen content is between 0.12 mmol O₂/g pollen/day stored and 0.57 mmol O₂/g pollen/day stored, inclusive. In another embodiment, the starting oxygen content is between 0.24 mmnol O₂/g pollen/day stored to 0.57 mmol O/g pollen/day stored, inclusive. “Starting oxygen content,” “Start mmol O₂,” “Start mmol O₂/g pollen,” and “Start mmol O₂/g pollen/day stored” are used interchangeably herein.

A “plant” is any plant at any stage of development, particularly a seed plant. In particular, in the context of this disclosure, a plant refers to a maize plant. As used herein, the term “plant line” refers to a single plant material or a genetically identical set of materials

“Platform,” as used herein, means a surface within a container which is in direct contact with the pollen and carrier mixture, and which prevents direct contact with the container itself. For example, the platform may be filter paper or an aluminum tray.

“Pollen:Carrier Ratio,” as used herein, means the proportion of pollen present in a mixture with a carrier. For example, and not by way of limitation, a mixture of pollen and carrier with a pollen:carrier ratio of 2:1 comprises two parts pollen measured by weight or volume and one part carrier compound, e.g., talc, measured by weight or volume.

“Refrigerated environment,” as used herein, means any condition where the temperature is less than ambient temperature (or room temperature), but does not fall below the temperature at which water freezes. Said another way, if ambient temperature is 25° C., then a refrigerated environment comprises temperatures greater than 0° C. and less than 25° C. Likewise, a refrigerated environment comprises temperatures between 2° C. and 10° C.

“Seed Set,” as used herein, means the number of kernels produced on a cob from a successful pollination. Seed set may be expressed qualitatively (e.g., low, good, or high) or quantitatively. In a quantitative measurement, the measurement may be given as either a percentage or a number of seeds per ear. The term generally refers to the percentage or number of normal kernels (i.e. non-aborted, endosperm-viable kernels). For normal maize lines (i.e. not haploid inducer lines), a seed set above 80% (or above 300 kernels per ear) is considered a good seed set. Achieving a good seed set is a goal of a controlled pollination.

“Storage,” as used herein, refers to the act of storing pollen for a suitable period. A suitable storage period may be as little as 24 hours or as much as 12 days.

“Treatment,” as used herein, means intentional application of compounds or environmental constraints to pollen. In particular, a pollen treatment may include addition of a carrier compound to the pollen to preserve the pollen's flowability and viability.

“Vessel pressure,” as used herein, refers to any artificially imposed atmospheric pressure within the vessel. Vessel pressure values are measured here in units of standard atmosphere “atm,” e.g., 0.5 atm, however, other units may be used (e.g., Torr or Pascal or “Pa;” 1 Pa=9.8692×10⁻⁶ atm) as desired to measure vessel pressure. It is expressly contemplated that vessel pressure may meet or exceed 1 atm. Under conditions where the vessel pressure is between 0 and 1 atm, “vessel pressure” and “vacuum” possess the same meaning and are used interchangeably. “Vessel pressure” is the preferred term, however, as it contemplates both vacuum conditions and conditions where the artificially imposed atmospheric pressure exceeds ambient atmosphere (e.g., 1 atm).

“Pollen viability” as used herein refers to the ability of a pollen grain to germinate a pollen tube. During a pollination event, this pollen tube would grow through a stigma (silk), deliver two sperm cells to the female gametophyte that would fertilize the egg cell and the two polar nuclei, which would result in the formation of an embryo and endosperm respectively.

“Pollen vigor” as used herein refers to the interval of time between when a pollen grain physically contacts a maize silk and when the subsequent fertilization takes place. Pollen vigor is critical for seed set, as a viable pollen grain must complete pollen tube growth through the stigma and subsequent fertilization before viability is lost due to desiccation or other environmental stress factors.

### DETAILED DESCRIPTION

Producibility in maize seed production (i.e., a measure of whether the required quantities of inbred or hybrid seed can be produced through self-pollination or cross pollination at an economical cost that does not exceed the value of the seed being produced) is a critical factor for success in developing maize inbred parent lines, as large quantities of inbred parent line seed are required to produce the hybrid seed sold to customers. A maize inbred parent line with low producibility may be discontinued due to excessive costs in parent seed production, even if that inbred parent line can produce hybrids with characteristics that are desirable to customers (e.g., leading GM and genome edited traits, high yield, disease resistance). Pollen storage technology can be used to enhance the producibility of inbred maize parent lines used in hybrid seed production.

Challenges to producibility that may be addressed by pollen storage technology include but are not limited to, low pollen production, low total pollen shed, short duration of pollen shed, short duration of silk receptivity, and GM or genome edited traits that may impact plant reproductive characteristics. An additional challenge with self-pollination may be a long self-split, which is defined by the number of days between when pollen starts shedding and when silks emerge and become available for pollination. In some iterations, self-split can be a negative value, where silks emerge for pollination before the start of pollen shed. The observed self-split may be a result of the inbred parent line genetics or a result of stress in the growing environment that reduces the rate of silk extension and increases the number of days between start of pollen shed and silk availability for pollination.

To address these producibility challenges, pollen storage technology may be used to collect pollen during the optimal window for pollen shed, store that pollen while maintaining pollen viability, then apply the pollen during the optimal window for silk emergence and receptivity. In some iterations, pollen collection may be conducted multiple times per day. In other iterations, pollen may be collected on multiple days throughout the duration of pollen shed. Application of stored pollen may combine pollen collected over multiple days and multiple applications may take place on the same day or across multiple days. Pollen application may combine pollen collected from multiple field locations into a single application to one location. In some iterations, pollen may be collected in one geography and applied to silks in a different geography. The geographies may be different fields at the same production location, fields in different states or municipalities within country, or fields in different countries. In some iterations, pollen is collected from temperate maize inbred parent lines grown in a temperate location and applied to sub-tropical or tropical maize inbred parent lines grown in sub-tropical or tropical locations. In other iterations, pollen is collected from sub-tropical or tropical maize inbred parent lines grown in sub-tropical or tropical locations and applied to temperate maize inbred parent lines grown in a temperate location. By addressing these challenges to producibility, pollen storage technology may enable seed increase for desirable maize inbred parent lines that will produce new hybrids with desirable characteristics for sale to customers. Pollen storage technology may also enable economical hybrid seed production for combinations of temperate, sub-tropical, and tropical maize inbred parent lines that are not currently feasible.

Accordingly, an embodiment provides a method of storing viable maize pollen, comprising collecting an amount of fresh maize pollen, optionally applying a carrier to the collected maize pollen, obtaining an amount of treated maize pollen, placing the amount of fresh maize pollen or the amount of treated maize pollen in a container, sealing the container with a breathable barrier, and storing the pollen in the container in a refrigerated environment. In one embodiment, the amount of fresh maize pollen or the amount of treated maize pollen is 0 days old, 1 day old, 2 days old, 3 days old, 4 days old, 5 days old, 6 days old, 7 days old, 8 days old, 9 days old, 10 days old, 11 days old, 12 days old, 13 days old, 14 days old, 15 days old, 16 days old, 17 days old, 18 days old, 19 days old, 20 days old, or more. In another embodiment, the amount of fresh maize pollen or treated maize pollen is about 0.3 grams to 10 kilograms and in another embodiment, the amount of fresh maize pollen or treated maize pollen is about 2 grams to 1 kilogram. In one embodiment, the carrier is selected from the group consisting of crystalline silica, talc, metallic powder, and mica minerals. In another embodiment, the carrier is crystalline silica and comprises an average particle size. The average particle size may be between about 1 nanometer and about 100 micrometers. In another embodiment the average particle size is 10 micrometers. In yet another embodiment, the metallic powder is metallic oxide powder or metallic carbide powder. In another embodiment, the metallic powder comprises an average particle size that may be between about 1 micrometer and about 100 micrometers. In one embodiment, the average particle size is about 10 micrometers spherical. In another embodiment, the metallic powder is stainless steel powder.

In another embodiment, the carrier is present in a pollen:carrier ratio selected from the group consisting of 1:20, 1:30, 1:10, 1:5, 1:3, 1:2, 1:1, 2:1, 3:1, 4:1, 5:1, 6:1, 7:1, 8:1, 9:1, 10:1, 20:1, 30:1, 40:1, 50:1, and any ratio between 1:20 and 50:1 The pollen:carrier ratio is preferable 2:1. In one embodiment, the container comprises a volume of 0.1 milliliters to 10 liters. In another embodiment, the container comprises a volume of 10 milliliters to 1500 milliliters. In a further embodiment, the container comprises a volume of 100 milliliters to 1250 milliliters. The container may further comprise a CO₂ sequestration agent selected from the group consisting of activated charcoal, ethanolamine, Zeolite 4A, lithium hydroxide (LiOH), soda lime, calcium silicate (Ca2O4Si), and activated magnesium silicate (e.g., FLORISIL®). In one embodiment, the sequestration agent is soda lime.

In one embodiment, the breathable barrier is selected from the group consisting of Parafilm, Tyvek, 3M Micropore tape, cellulose, nitrocellulose, and a non-airtight container. In another embodiment, the breathable barrier is 3M micropore tape. In another embodiment, the non-airtight container comprises an aperture for gas exchange. The aperture may also comprise at least one perforation. In a further embodiment, the aperture comprises at least one perforation having a diameter size of 0.10 millimeters to 30 millimeters. In an embodiment, the breathable barrier comprises a surface area of 0.49 mm² per gram of fresh or treated pollen to 47.5 mm² per gram of fresh or treated pollen. In another embodiment, the breathable barrier comprises a surface area of 1.98 mm² per gram of fresh or treated pollen to 26.72 mm² per gram of fresh or treated pollen. In another embodiment, the breathable barrier comprises a surface area of 4.45 mm² per gram of fresh or treated pollen to 11.88 mm² per gram of fresh or treated pollen.

The stored maize pollen may remain viable for at least 1, 2, 3, 4, 5, 6, 7, 8, 9, 10, 11, 12, 13, 14, 15, 16, 17, 18, 19, or 20 days. In an embodiment, the refrigerated environment comprises a temperature range selected from the group consisting of 1° C.-10° C., 4° C.-8° C., and 5.5° C.-6.5° C. In another embodiment, the refrigerated environment comprises a temperature of approximately 6° C. In a further embodiment, the pollen is stored in the refrigerated environment for 20 or fewer days, 19 or fewer days, 18 or fewer days, 17 or fewer days, 16 or fewer days, 15 or fewer days, 14 or fewer days, 13 or fewer days, 12 or fewer days, 11 or fewer days, 10 or fewer days, 9 or fewer days, 8 or fewer days, 7 or fewer days, 6 or fewer days, 5 or fewer days, 4 or fewer days, 3 or fewer days, 2 or fewer days, or 1 day, or less than 1 day. In one embodiment, the pollen is stored for 12 or fewer days.

In an embodiment, the maize pollen is transgenic maize pollen. In another embodiment, the transgenic maize pollen comprises a transgenic event selected from the group consisting of MIR162, 3272, Bt11, GA21, MIR604, MZIR098, 5307, DAS40278, TC1507, DAS-59122-7, NK603, MON810, MON863, MON89034, MON88017, DP-4114, and MON87411. In one embodiment, the maize pollen comprises transgenic events Bt11, GA21, and MIR162. In another embodiment, the maize pollen comprises transgenic events Bt11 and MIR162. In an embodiment, the maize pollen comprises transgenic event MIR162.

In yet another embodiment, an apparatus to store pollen is provided. Said apparatus comprises a vessel with a total breathable barrier surface area. In one embodiment, the vessel comprises multiple openings. In another embodiment, the vessel comprises a total breathable barrier surface area between 0.49 mm² per gram of fresh or treated pollen and 47.5 mm² per gram of fresh or treated pollen. The pollen storage vessel may comprise at least one opening, at least two openings, at least three openings, at least four openings, at least five openings, at least six openings, at least seven opening, at least eight openings, at least nine openings, at least ten openings, at least eleven openings, at least twelve openings, at least thirteen openings, at least fourteen openings, at least fifteen openings, at least sixteen openings, at least seventeen openings, at least eighteen openings, at least nineteen openings, at least twenty openings, at least twenty-one openings, at least twenty-two openings, at least twenty-three openings, at least twenty-four openings, at least twenty-five openings, at least twenty-six openings, at least twenty-seven openings, at least twenty-eight openings, at least twenty-nine openings, or at least thirty openings.

In an embodiment, an individual opening of the apparatus has a total surface area of 15.2 mm² to 660.5 mm. The individual opening is selected from the group consisting of a circle, an oval, a square, a rectangle, a triangle, and any other two-dimensional shape. In one embodiment, the opening is a circle. In another embodiment, the vessel comprises twenty-two openings. The twenty-two openings may be circular. In a separate embodiment, the vessel comprises twenty-eight openings. The twenty-eight openings may be circular. In an embodiment, the twenty-eight total circular openings have a total surface area of 2199.1 mm.

In an embodiment, the multiple openings comprise a total combined surface area ranging between 0.49 mm² per gram of fresh or treated pollen and 47.5 mm² per gram of fresh or treated pollen. The multiple openings may be circular holes 5-15 mm in diameter. The multiple openings may also be circular holes 10 mm in diameter. In another embodiment, the circular holes have an individual surface area of 78.5 mm².

In an embodiment, the multiple openings are individually covered in a breathable barrier, wherein the multiple openings are distributed such that no pollen grain is greater than 47 mm from the nearest breathable barrier, or any other distribution that optimizes the farthest possible distance between pollen grains and the nearest breathable barrier. In an embodiment, the breathable barrier is selected from the group consisting of Parafilm, Tyvek, 3M Micropore tape, cellulose, nitrocellulose, and a non-airtight container. The breathable barrier may be 3M Micropore tape.

In an embodiment, the pollen storage vessel is a VWR® Cell Culture Flask with the vented cap used in its stock configuration with the nitrocellulose membrane in the vented cap acting as a breathable barrier. In another embodiment, vessel is a VWR® Cell Culture Flask used in its stock configuration with the cap removed and the vessel mouth covered in 3M micropore tape to act as a breathable barrier. In another embodiment, the vessel is a VWR® Cell Culture Flask modified to include 28 additional openings distributed over the surface of the vessel where the additional openings are covered in a breathable barrier and the stock, unvented cap is kept sealed over the vessel mouth. In another embodiment, the vessel is a VWR® Cell Culture Flask modified to include any number of additional openings covered with a breathable barrier in any distribution over the surface of the vessel a solid or vented cap left in place over the vessel mouth, or the cap removed and replaced with a breathable barrier. In yet another embodiment, the vessel is any brand of cell culture flask in a stock or modified configuration. The vessel may be any plastic, metal, or ceramic container with one or more openings for breathable barriers.

### Examples

### 1. Collection

Maize plants were grown in field and in greenhouse conditions. Once tassels emerged and had begun shedding pollen, bags were placed over the tassels to collect the pollen. Bags were typically placed during the late afternoon and removed the following morning. Collected pollen, after sifting away any anthers or other tassel material and optionally mixed with a carrier, was then placed in an appropriate, sealed container.

Alternatively, pollen is collected by harvesting the pre-shed tassels from the maize plants. The tassels can be placed in a beaker of water and allowed to shed pollen normally, or the tassels can be dried, macerated, and filtered to collect the pollen mechanically. See, e.g., U.S. Pat. No. 8,252,988 (filed Jun. 27, 2007), incorporated by reference herein in its entirety.

2. Experiments where a Mixture of Pollen and Talc is Stored in a Magenta GA7 Box with Added Soda Lime and Sealed with Parafilm as a Breathable Barrier.

Experiments 1, 2, and 3: 1) Breathable barrier (parafilm around GA7 box) with added soda lime (5 days); 2) Breathable barrier (parafilm around GA7 box) with added soda lime (5 days), and a larger starting pollen quantity than experiment 1; 3) Breathable barrier (parafilm around GA7 box) with added soda lime (5 days), and a larger starting pollen quantity than experiment 2.

For experiment 1, 2.522 g of pollen was mixed with 1.26 g of talc for a total of 3.783 g pollen mix with an initial fresh pollen talc mix moisture content (“PMC”) of 36.71%. Pollen was mixed at a ratio of two parts pollen, one part carrier by weight. A total of 3.513 g pollen mix (6.5 ml) was added to a 400 mL Magenta GA7 Box containing 0.44 g of soda lime. For experiment 2, compared to experiment 1 above, a larger amount of pollen (5.998 g) was mixed with 2.999 g talc (2:1 pollen:talc by weight) for a total of 9.997 g pollen mix with an initial fresh mix PMC of 34.16%. A total of 8.1 g of pollen mix (15 ml) was added to a 400 mL Magenta GA7 Box containing 1.02 g of soda lime. For experiment 3, a larger amount of pollen (23.89 g) than was used in experiments 1 and 2 was mixed with 11.944 g of talc (2:1 pollen:talc by weight) for a total of 35.82 g pollen mix with an initial fresh mix PMC of 35.97%. A total of 29.7 g of pollen mix (55 ml) was added to a 400 mL Magenta GA7 Box containing 3.74 g of soda lime. In all 3 experiments, soda lime was placed at the bottom of the GA7 box and contained in a 50 ml conical tube cap. The pollen mix was contained inside the base of a polylactic acid plastic (PLA) cup (˜45 mm×30 mm) and positioned above the open soda lime container. An iButtonLink DS1923-F5 #DS1923-F5 # Hygrochron Temperature and humidity data logger was placed above the pollen mix container to monitor internal relative humidity (rH) of the pollen storage vessel. The GA7 lid was placed on top of the GA7 box and gas exchange gap between box and lid was sealed by wrapping a single layer of 25.4 mm (1-inch) wide parafilm around the opening. The parafilm was stretched as is normal practice in plant tissue culture to create a tight seal. The GA7 lid to box gas exchange gap surface area was measured and calculated to total 42 mm². The pollen storage vessel was stored at 6° C. at 1 atm pressure for 5 days. After 5 days, pollen viability, final PMC relative to initial PMC, and end point gas analysis of O₂ and CO₂ was assessed.

In all three experiments, the iButtonLink DS1923-F5 # Hygrochron temperature and humidity data logger indicated a saturated rH above 98% was obtained throughout the duration of storage inside the storage vessel. For experiment 1, six replicate pollinations were made onto receptive silks of primary ears on hybrid maize plants. Pollen viability was 62% after 5 days of storage and the relative PMC was 96.3%. In experiment 2, six replicate pollinations were made onto receptive silks of primary ears on hybrid maize plants. Pollen viability was 66% after 5 days of storage and the relative PMC was 99.6%. In experiment 3, twelve replicate pollinations were made onto receptive silks of primary ears on hybrid maize plants. Pollen viability was 87% after 5 days of storage and increased significantly compared to fresh viability. The relative PMC was 100.9%. The internal vessel rH and the final relative PMC data indicate that the use of parafilm as a breathable barrier to seal the exposed 42 mm² surface area of the 400 mL Magenta GA7 Box reduced moisture loss from the storage vessel and the pollen mix. An internal gas sample acquired from the storage vessel after wrapping the gas exchange gap between box and lid with an air impermeable tape indicated an O₂ content of 19.7% and CO₂ content of 0.1% in experiment 1, O₂ content of 19.5% and CO₂ content of 0.1% in experiment 2, and O₂ content of 16.2% and CO₂ content of 0.3% in experiment 3. Use of parafilm as a breathable barrier provided sufficient gas exchange while minimizing water vapor transmission. The presence of soda lime provided sufficient sequestration of CO₂ in conjunction to the breathable properties of the barrier. An average seed set of 480 kernels indicate that the pollen was viable after 5 days of storage in experiment 1. An average seed set of 347 kernels indicate the pollen was viable after 5 days of storage in experiment 2. Experiment 2, compared to experiment 1, indicates the breathable barrier with soda lime allows for storage of a greater amount of pollen and talc while maintaining sufficient gas exchange and minimal water vapor transmission. The O₂ content in experiment 3 was lower than previously observed for experiments 1 and 2 conducted with smaller pollen mix amounts, but sufficient O₂ was available to support pollen respiration. An average seed set of 598 kernels indicate the pollen was viable after 5 days of storage for experiment 3. The high seed set indicates that large quantities of pollen and talc mixes can be stored in a container with the use of a breathable barrier.

TABLE 1
Pollen viability and gas composition
analysis for experiments 1, 2, and 3.

| | | Pollen | Final PMC | | |
|---|---|---|---|---|---|
| | | Tube | Relative to | % O₂ at | % CO₂ |
| | Days | Gemination | Initial | 1 atm, | at 1 atm, |
| Experiment | Stored | (%) | PMC (%) | 22° C. | 22° C. |
| 1 | 0 | 70 | NA | NA | NA |
| 1 | 5 | 62 | 96.3 | 19.7 | 0.1 |
| 2 | 0 | 78 | NA | NA | NA |
| 2 | 5 | 66 | 99.6 | 19.5 | 0.1 |
| 3 | 0 | 61 | NA | NA | NA |
| 3 | 5 | 87 | 100.9 | 16.2 | 0.3 |

NA = Not applicable

TABLE 2
Average seed set for experiments 1, 2, and 3.

| | Days | Replicate | Avg Seed | |
|---|---|---|---|---|
| Experiment | Stored | Pollinations | Set/Ear (k) | Stdev (k) |
| 1 | 5 | 6 | 480 | 74 |
| 2 | 5 | 6 | 348 | 124 |
| 3 | 5 | 12 | 599 | 102 |

Experiments 4 and 5: 4) Experiments where a mixture of pollen and talc is stored in a Magenta GA7 box sealed with parafilm as a breathable barrier with and without added soda lime.

For experiment 4, 11.362 g of pollen was mixed with 5.681 g of talc (2:1 pollen:talc by weight) for a total of 17.043 g pollen mix with an initial fresh mix PMC of 35.31%. Two treatments were prepared to compare protocol performance in storage containers with and without added soda lime. This comparison was carried out to determine if CO₂ would escape through the parafilm breathable barrier throughout the duration of storage. A total of 5.4 g of pollen mix (10 ml) was added to a 400 mL Magenta GA7 Box for each treatment. In treatment 1, 0.68 g of soda lime contained in a 50 ml conical tube cap was placed at the bottom of the GA7 box. In treatment 2, an empty 50 ml conical tube cap was placed at the bottom of a 400 mL Magenta GA7 Box.

For experiment 5, 30.527 g of pollen was mixed with 15.264 g talc (2:1 pollen:talc) for a total of 45.791 g pollen mix with an initial fresh mix PMC of 34.96%. A total of 8.1 g pollen mix (15 ml) was added to a 400 mL Magenta GA7 Box and an empty 50 ml conical tube cap was placed at the bottom with no added soda lime. The pollen mix was contained inside a PLA plastic cup (45 mm×30 mm) and positioned above the open 50 ml conical tube cap containing soda lime or above the empty 50 ml conical tube cap in all treatments. An iButtonLink DS1923-F5 # Hygrochron Temperature and humidity data logger was placed above the pollen mix container to monitor internal relative humidity of the pollen storage vessel. The GA7 lids were placed on top of the GA7 boxes and the gas exchange gap between box and lid was wrapped in a single layer of 25.4 mm (1-inch) wide parafilm. The parafilm was stretched as is normal practice in plant tissue culture to create a tight seal. The GA7 lid to box gas exchange gap surface area was measured and was calculated to total 42 mm². The pollen storage vessels were then stored at 6° C. at 1 atm pressure for 5 days. After 5 days, pollen viability, final PMC relative to initial PMC, and end point gas analysis of O₂ and CO₂ was assessed.

In experiment 4, nine replicate pollinations were made onto receptive silks of primary ears on hybrid maize plants for both treatments. Pollen viability was qualitatively scored a ranking of 4 (41-60% germination rate; see Table 29) after 5 days of storage and both treatments scored similarly. Treatment 1, with added soda lime, had a relative PMC of 105.6% and treatment 2, with no added soda lime, had a relative PMC of 101.4%. The final relative PMC data indicated that the use of parafilm as a breathable barrier to seal the 42 mm² gas exchange surface area of the 400 mL Magenta GA7 box reduced moisture loss from the storage vessel and pollen mix. For both treatments, moisture content of the pollen mix increased over time due to additional new water generated from cellular respiration. An internal gas sample was acquired from each treatment after wrapping the gas exchange gap with an air impermeable tape. Treatment 1 had an endpoint O₂ content of 19.2% and CO₂ content of 0.2%. Treatment 2 had an endpoint O₂ content of 19.5% and CO₂ content of 1.2%. The CO₂ content of Treatment 2 (no added soda lime) was higher than Treatment 1 (with added soda lime), but a CO₂ content of 1.2% demonstrated that CO₂ gas exchange occurred through the parafilm breathable barrier. Use of parafilm as a breathable barrier provided sufficient gas exchange while minimizing water vapor transmission. The presence of soda lime provided sufficient sequestration of CO₂ in conjunction to the breathable properties of the barrier. The breathable properties of the barrier provided sufficient gas exchange of CO₂ and O₂ to avoid toxic levels of CO₂ accumulation. These results suggest that soda lime may not be required for pollen storage if a breathable barrier is available for gas exchange. An average seed set of 381 kernels/ear for treatments with added soda lime and 426 kernels/ear for treatments no added soda lime indicates that the pollen in both treatments was viable after 5 days of storage. A higher seed set for the treatment without soda lime further supports that soda lime may not be required for pollen storage if a breathable barrier is available for gas exchange.

In experiment 5, three replicate pollinations were made onto receptive silks of primary ears on hybrid maize plants. The iButtonLink DS1923-F5 # Hygrochron temperature and humidity data logger indicated a saturated rH above 98% was obtained throughout the duration of storage inside the storage vessel. Pollen viability was annotated at 66% after 5 days of storage and the relative PMC was 102.1%. The internal vessel rH and the final relative PMC data indicate that the use of parafilm as a breathable barrier to seal the exposed 42 mm² gas exchange surface area of the 400 mL Magenta GA7 box reduced moisture loss from the storage vessel and pollen mix. Moisture content of the pollen mix increased over time due to additional new water generated by cellular respiration. An internal gas sample acquired from the storage vessel after wrapping the gas exchange gap with an air impermeable tape indicated an O₂ content of 19.6% and CO₂ content of 0.9%. Use of parafilm as a breathable barrier provided sufficient gas exchange while minimizing water vapor transmission. CO₂ levels remained below 1.0% and toxic levels of CO₂ accumulation were avoided, suggesting soda lime may not be required for this approach to pollen storage. An average seed set of 514 kernels/ear indicate that the pollen was viable after 5 days of storage.

TABLE 3
Pollen viability assessment and gas composition
analysis for treatments in experiment 4.

| | | Final PMC | | |
|---|---|---|---|---|
| | | Relative to | % O₂ at | % CO₂ |
| Days | | Initial PMC | 1 atm, | at 1 atm, |
| Stored | Treatment | (%) | 22° C. | 22° C. |
| 5 | (+) soda lime | 105.6 | 19.2 | 0.2 |
| 5 | (−) soda lime | 101.4 | 19.5 | 1.2 |

TABLE 4
Average seed set (experiment 4)

| Days | | Replicate | Avg Seed | |
|---|---|---|---|---|
| Stored | Treatment | Pollinations | Set/Ear (k) | Stdev (k) |
| 5 | (+) soda lime | 9 | 381 | 168 |
| 5 | (−) soda lime | 9 | 427 | 139 |

TABLE 5
Pollen viability assessment and gas
composition analysis (experiment 5)

| | Pollen Tube | Final PMC | | |
|---|---|---|---|---|
| Days | Gemination | Relative to | % O₂ at 1 | % CO₂ at 1 |
| Stored | (%) | Initial PMC (%) | atm, 22° C. | atm, 22° C. |
| 0 | 68 | NA | NA | NA |
| 5 | 66 | 102.1 | 19.6 | 0.9 |

NA = Not Applicable

TABLE 6
Average seed set (experiment 5)

| Days | Replicate | Avg Seed | |
|---|---|---|---|
| Stored | Pollinations | Set/Ear (k) | Stdev (k) |
| 5 | 3 | 514 | 95 |

3. Experiments where a Mixture of Pollen and Crystalline Silica is Stored in a Magenta GA7 Box with Added Soda Lime.

### Experiment 1: Breathable Barrier (Parafilm Around GA7 Box) with Crystalline Silica and Soda Lime (7 Days)

Pollen in the amount of 5.955 g was mixed with 2.9775 g of 10 μm crystalline silica (2:1 pollen:silica by weight) for a total of 8.933 g pollen mix with an initial fresh mix PMC of 33.57%. A total of 8.85 g pollen mix (12.5 ml) was added to a 400 mL Magenta GA7 box containing 1.02 g of soda lime. The soda lime was placed at the bottom of the GA7 box and was contained in a 50 ml conical tube cap. The pollen mix was contained inside a PLA plastic cup (45 mm×30 mm) and positioned above the open soda lime container. An iButtonLink DS1923-F5 # Hygrochron Temperature and humidity data logger was placed above the pollen mix container to monitor internal relative humidity of the pollen storage vessel. The GA7 lid was placed on top of the GA7 box and the gas exchange gap between box and lid was sealed by wrapping a single layer of 25.4 mm (1-inch) wide parafilm around the open gap. The parafilm was stretched as is normal practice in plant tissue culture to create a tight seal. The GA7 gas exchange gap between box and lid surface area is calculated to total 42 mm². The pollen storage vessel was stored at 6° C. at 1 atm pressure for 7 days. After 7 days, pollen viability, final PMC relative to initial PMC, and end point gas analysis of O₂ and CO₂ was assessed.

Four replicate pollinations were made onto receptive silks of primary ears on hybrid maize plants. The iButtonLink DS1923-F5 # Hygrochron temperature and humidity data logger indicated a saturated rH above 98% was obtained throughout the duration of storage inside the storage vessel. Pollen viability was qualitatively scored a ranking of 4 (41-60% germination rate; see Table 29) after 7 days and the relative PMC was 104.9%. The internal vessel rH and the final relative PMC data indicate that the use of parafilm as a breathable barrier reduced moisture loss from the storage vessel and pollen mix. Moisture content of the pollen mix increased over time due to additional water generated by cellular respiration. An internal gas sample acquired from the storage vessel after wrapping the seal with an air impermeable tape indicated an O₂ content of 19.4% and CO₂ content of 0.1%. Use of parafilm as a breathable barrier provided sufficient gas exchange while minimizing water vapor transmission. The presence of soda lime provided sufficient sequestration of CO₂ in conjunction to the breathable barrier. An average seed set of 670 kernels/ear indicate that the pollen was viable after 7 days of storage.

TABLE 7
Final PMC assessment and gas composition
analysis for experiment 1.

| | Final PMC Relative | % O₂ at 1 | % CO₂ at 1 |
|---|---|---|---|
| Days Stored | to Initial PMC (%) | atm, 22° C. | atm, 22° C. |
| 7 | 104.9 | 19.4 | 0.1 |

TABLE 8
Average seed set for experiment 1.

| | Replicate | Avg Seed | |
|---|---|---|---|
| Days Stored | Pollinations | Set/Ear (k) | Stdev (k) |
| 7 | 4 | 670 | 38 |

Experiments 2 and 3: 2) Breathable barrier (parafilm around a GA7 box) with crystalline silica, soda lime, and a reduced headspace (5 day); 3) Breathable barrier (parafilm around a GA7 box) with crystalline silica, soda lime, a reduced headspace, and a larger starting quantity of pollen than experiment 2 (5 day).

In experiment 2, 23.623 g of pollen was mixed with 11.812 g of 10 μm crystalline silica (2:1 pollen:silica ratio by weight) for a total of 35.4345 g pollen mix with an initial fresh mix PMC of 35.62%. A total of 8.5 g of pollen mix (12.5 ml) was added to each of four Magenta GA7 box treatments. Three Magenta GA7 boxes were modified by partially filling the box with an epoxy resin to achieve final headspace volumes of 100 ml, 200 ml, and 300 ml as needed. Epoxy resin was allowed to cure in a pressure chamber set to 3 atm absolute for a minimum of 24 hours prior to use in pollen storage. A fourth box (400 ml headspace) was used as a positive control. 1.05 g of soda lime was contained in a 50 ml conical tube cap and placed into the bottom of each box for the 200 ml, 300 ml, and 400 ml headspace treatments. The soda lime was placed directly on top of the cured epoxy resin in the 100 ml headspace treatment but was kept separated from the container of pollen:silica mix.

In experiment 3, 10.484 g of pollen was mixed with 5.242 g of 10 μm crystalline silica (2:1 pollen:silica ratio) for a total of 15.726 g pollen mix with an initial fresh mix PMC of 35.93%. A total of 15.72 g pollen mix (22 ml) was added to a modified Magenta GA7 box. The box was modified by partially filling the box with an epoxy resin to achieve a final headspace volume of 100 ml. 1.87 g of soda lime was placed directly on top of the cured epoxy resin but was separated from the pollen:silica mix container. For both experiments, the pollen mix was contained inside a PLA plastic cup (45 mm×30 mm) and positioned above the open soda lime container or beside the soda lime placed directly on top of the cured epoxy resin. For each treatment, the GA7 lid was placed on top of the GA7 box and the gas exchange gap between box and lid was sealed by wrapping a single layer of 25.4 mm (1-inch) wide parafilm around the opening. The parafilm was stretched as is normal practice in plant tissue culture to create a tight seal. The GA7 gas exchange gap between box and lid surface area is calculated to total 42 mm². The pollen storage vessels were stored at 6° C. at 1 atm pressure for 5 days. After 5 days of storage, pollen viability, final PMC relative to initial PMC, and end point gas analysis of O₂ and CO₂ was assessed.

For experiment 2, three replicate pollinations per treatment were made onto receptive silks of primary ears on hybrid maize plant. Pollen viability was qualitatively scored a ranking of 5 (>60% germination rate; see Table 29) after 5 days of storage for all treatments. The relative PMC ranged from 99.0% to 104.8% across treatments. An internal gas sample was acquired for each treatment from the storage vessel after wrapping the gas exchange gap with an air impermeable tape. O₂ contents ranged from 18.6 to 19.3% across the treatments and CO₂ contents were 0.1%.

For experiment 3, six replicate pollinations were made onto receptive silks of primary ears on hybrid maize plants. The relative PMC was 97.4%. An internal gas sample was acquired from the storage vessel after wrapping the gas exchange gap with an air impermeable tape. The O₂ content was 19.6% and CO₂ content was 0.1%. In both experiments, the use of parafilm as a breathable barrier provided sufficient gas exchange while minimizing water vapor transmission. The presence of soda lime provided sufficient sequestration of CO₂ in conjunction with the breathable barrier.

The reduction of headspace from 400 ml down to 100 ml in experiment 2 and experiment 3 demonstrated that the initial starting quantity of moles of O₂ present in the vessel volume does not limit pollen respiration with the provided 42 mm² surface area of parafilm breathable barrier covering the gas exchange gap in each GA7 Magenta box. O₂ is constantly replenished into the pollen storage vessel while moisture loss is limited. In experiment 2, an average seed set for the 400 ml, 300 ml, 200 ml, and 100 ml headspace storage containers was 535, 510, 502 and 668 kernels/ear, respectively, indicating that the pollen was viable after 5 days of storage. In experiment 3, an average seed set of 564 kernels/ear indicated the same conclusion.

TABLE 9
Final PMC assessment and gas composition analysis for experiments 2 and 3.

| | | | Final PMC | | | Grams of |
|---|---|---|---|---|---|---|
| | | GA7 Magenta | Relative | % O₂ | % CO₂ | pure pollen |
| | Days | box headspace | to Initial | at 1 atm, | at 1 atm, | per liter |
| Experiment | Stored | (ml) | PMC (%) | 22° C. | 22° C. | headspace (g) |
| 2 | 5 | 400 | 104.8 | 19.1 | 0.1 | 14.76 |
| 2 | 5 | 300 | 100.6 | 19.2 | 0.1 | 19.69 |
| 2 | 5 | 200 | 99.0 | 18.6 | 0.1 | 29.53 |
| 2 | 5 | 100 | 100.6 | 19.3 | 0.1 | 59.06 |
| 3 | 5 | 100 | 97.4 | 19.6 | 0.1 | 104.8 |

TABLE 10
Average seed set for experiments 2 and 3.

| | | Magenta | | | |
|---|---|---|---|---|---|
| | | GA7 box | | | |
| | Days | headspace | Replicate | Avg Seed | |
| Experiment | Stored | (ml) | Pollinations | Set/Ear (k) | Stdev (k) |
| 2 | 5 | 400 | 3 | 536 | 42 |
| 2 | 5 | 300 | 3 | 510 | 93 |
| 2 | 5 | 200 | 3 | 502 | 89 |
| 2 | 5 | 100 | 3 | 668 | 65 |
| 3 | 5 | 100 | 6 | 565 | 70 |

### Experiment 4: Reduction of Breathable Barrier Surface Area in Magenta GA7 Boxes Containing Pollen Mixed with Crystalline Silica with Added Soda Lime

To evaluate the reduction of breathable barrier surface area, 24.871 g of pollen was mixed with 12.436 g of 10 μm crystalline silica (2:1 pollen:silica) for a total of 37.307 g pollen mix with an initial fresh mix PMC of 34.94%. A total of 9.2 g of pollen mix was added to each of four 400 ml Magenta GA7 boxes. Three of the four boxes were modified by sealing the gas exchange gap between box and lid with modeling clay to seal off gas exchange on one, two, or three sides of the lid, respectively. The sealed edges effectively reduced the surface area available for gas exchange to 10.5 mm², 21 mm², and 31.5 mm². A fourth box with the same 400 ml headspace and without any gas exchange gaps sealed with modeling clay was used as a positive control (surface area of 42 mm²). A 50 ml conical tube lid containing 1.11 g of soda lime was placed at the bottom of each box. The pollen mix was contained inside of a PLA plastic cup (45 mm×30 mm) and positioned above the open soda lime container. The GA7 lid was placed on top of the GA7 box and the gas exchange gap between box and lid was sealed by wrapping a single layer of 25.4 mm (1-inch) wide parafilm around the opening for each treatment. The parafilm was stretched as is normal practice in plant tissue culture to create a tight seal. The pollen storage vessels were stored at 6° C. at 1 atm pressure for 5 days. After 5 days of storage, pollen viability, final PMC relative to initial PMC, and end point gas analysis of O₂ and CO₂ was assessed for each treatment.

Pollen viability ranged from 46% to 50% after 5 days of storage and the relative PMC ranged from 96.9 to 99.9% across treatments. As the breathable barrier surface area decreased, the final PMC increased. An internal gas sample was acquired for each treatment from the storage vessel after wrapping the gas exchange gap between box and lid with an air impermeable tape. O₂ contents ranged from 17.8 to 19.1% across treatments and CO₂ contents were all 0.1%. As breathable barrier surface area decreased, so did the final O₂ content. Use of reduced surface area covered by the parafilm breathable barrier provided for sufficient gas exchange while minimizing water vapor transmission down to 10.5 mm². The presence of soda lime provided sufficient sequestration of CO₂ in conjunction to the breathable barrier.

TABLE 11
Pollen viability assessment and gas composition
analysis for experiment 4.

| | Total | | | | |
|---|---|---|---|---|---|
| | Breathable | | Final | | |
| | Membrane | Pollen | PMC | % O₂ | % CO₂ |
| | Surface | Tube | Relative to | at 1 | at 1 |
| Days | Area | Gemination | Initial PMC | atm, | atm, |
| Stored | (mm²) | (%) | (%) | 22° C. | 22° C. |
| 0 | NA | 60 | NA | NA | NA |
| 5 | 10.5 | 50 | 99.9 | 17.8 | 0.1 |
| 5 | 21 | 49 | 99.6 | 18.2 | 0.1 |
| 5 | 31.5 | 50 | 96.9 | 19.0 | 0.1 |
| 5 | 42 | 46 | 97.0 | 19.1 | 0.1 |

NA = Not Applicable

TABLE 12
Final vessel oxygen content for four
breathable membrane surface areas.

| Breathable Barrier Surface | % O₂ at 1 |
|---|---|
| Area (mm²) | atm, 22° C. |
| 10.5 | 17.8 |
| 21 | 18.2 |
| 31.5 | 19.0 |
| 42 | 19.1 |

### 4. Alternative Storage Container—Plastic Clamshell Containers

Experiments 1 and 2: 1) Plastic clamshell box as a breathable barrier (i.e., non-airtight container with a gap between sections) with crystalline silica and added soda lime—no pollinations (5 day); 2) Plastic clamshell box as a breathable barrier (i.e., non-airtight container with a gap between sections) with crystalline silica and soda lime—with pollinations (5 day)

In experiment 1, 11.609 g of pollen was mixed with 5.805 g of 10 μm crystalline silica (2:1 pollen:silica by weight) for a total of 17.414 g pollen mix with an initial fresh mix PMC of 33.82%. A total of 17.00 g pollen mix was added to a 630 ml polyethylene terephthalate glycol clear plastic clamshell Phytatray™ brand container. 2.07 g of soda lime contained in a 50 ml conical tube cap was placed at the bottom of the clamshell container. In experiment 2, 19.876 g of pollen was mixed with 9.938 g of 10 μm crystalline silica (2:1 pollen:silica) for a total of 29.814 g pollen mix with an initial fresh mix PMC of 35.9%. A total of 22.3 g pollen mix was added to the 630 ml clear deep plastic clamshell container and 3.15 g of soda lime was added directly to the bottom of the container. For both experiments, the pollen mix was contained inside of a PLA plastic cup (45 mm×30 mm) and positioned next to the open soda lime container or loose soda lime. An iButtonLink DS1923-F5 # Hygrochron temperature and humidity data logger was placed next to the pollen mix container to monitor internal relative humidity of the pollen storage vessel. The clamshell lid was placed on top of the clamshell bottom and snapped into place. Any gap present between the two clamshell container sections was not sealed with any form of barrier. The pollen storage vessel was then stored at 6° C. at 1 atm pressure for 5 days. After 5 days of storage, pollen viability, final PMC relative to initial PMC, and end point gas analysis of O₂ and CO₂ was assessed.

In experiment 1, the iButtonLink DS1923-F5 # Hygrochron temperature and humidity data logger indicated a saturated rH above 98% was obtained throughout the duration of storage inside the storage vessel. Pollen viability was 51% after 5 days of storage and the relative PMC was 95.8%. The internal vessel rH and the final relative PMC data indicate that the clamshell container with sections sealed together using only the manufactured locking mechanism functioned as a breathable barrier. An internal gas sample acquired from the storage vessel indicated an O₂ content of 19.8% and CO₂ content of 0.1%. The clamshell container provided sufficient gas exchange while minimizing water vapor transmission, while the presence of soda lime provided sufficient sequestration of CO₂ in conjunction with the gap between clamshell sections acting as a breathable barrier. The clamshell breathable barrier provided for comparable pollen tube germination (51%) to the fresh pollen mix control (54%).

For experiment 2, four replicate pollinations were made onto receptive silks of primary ears on hybrid maize plants. Pollen viability was qualitatively scored a ranking of 3 (>21 to 40% germination rate; see Table 29) after 5 days of storage and the relative PMC was 99.3%. An internal gas sample acquired from the storage vessel indicated an O₂ content of 19.9% and CO₂ content of 0.1%. The clamshell container provided sufficient gas exchange while minimizing water vapor transmission, while the presence of soda lime provided sufficient sequestration of CO₂ in conjunction with the gap between clamshell sections acting as a breathable barrier. An average seed set of 784 kernels/ear indicated that the pollen was viable after 5 days of storage.

TABLE 13
Pollen viability assessment and gas composition analysis for experiments 1 and 2.

| | | | Pollen | Final PMC | | |
|---|---|---|---|---|---|---|
| | | | Tube | Relative to | % O₂ | % CO₂ |
| | Days | | Gemination | Initial | at 1 atm, | at 1 atm, |
| Experiment | Stored | Treatment | (%) | PMC (%) | 22° C. | 22° C. |
| 1 | 5 | 630 ml clamshell | 51 | 96 | 19.8 | 0.1 |
| 2 | 5 | 630 ml clamshell | ND | 99 | 19.9 | 0.1 |

ND = No data

TABLE 14
Average seed set for experiment 2.

| | Days | | Replicate | Avg Seed | |
|---|---|---|---|---|---|
| Experiment | Stored | Treatment | Pollinations | Set/Ear (k) | Stdev (k) |
| 2 | 5 | 630 ml | 4 | 728 | 117 |
| | | clamshell | | | |

### 5: Tyvek, Micropore Tape, and Open Perforations Breathable Barrier Surface Area Applied to an Otherwise Airtight Container.

In experiments 1, 2, 3, and 6 in Example 4, Tyvek was used to allow for gas exchange while reducing water vapor transmission out of the pollen storage container. In experiments 4, 5, 6, and 7, Micropore Tape was evaluated to allow for gas exchange while reducing water vapor transmission out of the pollen storage container in the same manner as Tyvek. In experiments 1-7 and 9 in example 4, 100 ul of H₂O was added to the bottom of storage containers to account for low humidity in the testing environment (30% rH). The addition of the water helped the internal atmosphere of the storage containers reach a saturated relative humidity.

### Experiment 1: Tyvek as an Alternative Breathable Barrier with Added Soda Lime (5 Day)

To evaluate Tyvek as a breathable barrier, 9.7551 g of pollen was mixed with 4.8776 g of 10 μm crystalline silica (2:1 pollen:silica by weight) for a total of 14.633 g pollen mix with an initial fresh mix PMC of 37.02%. A total of 14.0 g pollen mix was added to a 500 ml wide mouth mason jar. A PLA plastic cup containing 1.73 g of soda lime was added to the 500 ml wide mouth mason jar. The pollen mix was contained inside an aluminum pan (45 mm×35 mm). The pollen mix pan was stacked above the soda lime cup and separated by a metal 841 micrometer mesh screen. A wide mouth lid was equipped with a septum for gas analysis and a 3.175 mm perforation was made in the lid with a handheld die press to act as a breathable barrier. A disk of Tyvek was cut from a standard FedEx envelope and was taped over the 3.175 mm perforation (⅛-inch). An impermeable plate seal film was applied to the outer perimeter of the Tyvek to seal the Tyvek to the container lid. The 3.175 mm (⅛-inch) perforation represents a 7.9 mm² breathable barrier surface area. An iButtonLink DS1923-F5 # Hygrochron temperature and humidity data logger was stacked above the pollen mix container to monitor internal relative humidity of the pollen storage vessel. The iButtonLink DS1923-F5 # Hygrochron temperature and humidity data logger was separated from the pollen mix container using a metal 841 micrometer mesh screen. The pollen storage vessel lid was sealed with a mason jar lid band and stored at 6° C. at 1 atm pressure for 5 days. After 5 days of storage, pollen viability, final PMC relative to initial PMC, and end point gas analysis of O₂ and CO₂ was assessed.

Two replicate pollinations were made onto receptive silks of primary ears on hybrid maize plants. Pollen viability qualitatively scored a ranking of 3 (>21 to 40% germination rate; see Table 29) after 5 days of storage and the relative PMC was 102.5%. An internal gas sample acquired from the storage vessel indicated an O₂ content of 19.1% and CO₂ content of 0.1%. The Tyvek covered 3.175 mm (⅛-inch) perforation provided sufficient gas exchange while minimizing water vapor transmission. The presence of soda lime provided sufficient sequestration of CO₂ in conjunction to the breathable barrier. The iButtonLink DS1923-F5 # Hygrochron temperature and humidity data logger indicated a saturated rH above 98% was obtained throughout the duration of storage inside the storage vessel. An average seed set of 789 kernels/ear indicate the pollen was viable after 5 days of storage.

TABLE 15
Pollen viability assessment and gas composition analysis

| | | Breathable | Final | | |
|---|---|---|---|---|---|
| | | Barrier | PMC | | |
| | Breathable | Surface | Relative to | | |
| Days | Barrier | Area | Initial | % O₂ at 1 | % CO₂ at 1 |
| Stored | Treatment | (mm²) | PMC (%) | atm, 22° C. | atm, 22° C. |
| 5 | Tyvek | 7.9 | 102.5 | 19.1 | 0.1 |

TABLE 16
Average seed set

| | | Breathable | | | |
|---|---|---|---|---|---|
| | | Barrier | | | |
| | Breathable | Surface | | | |
| Days | Barrier | Area | Replicate | Avg Seed | |
| Stored | Treatment | (mm²) | Pollinations | Set/Ear (k) | Stdev (k) |
| 5 | Tyvek | 7.9 | 2 | 789 | 30 |

### Experiment 2: Tyvek Breathable Barrier Surface Area Factorial with Added Soda Lime

In this experiment, 22.608 g of pollen was mixed with 11.304 g of 10 μm crystalline silica (2:1 pollen:silica by weight) for a total of 33.912 g pollen mix with an initial fresh mix PMC of 34.08%. A total of 4.0 g pollen mix (2.67 g pollen) was added to each of seven 125 ml regular mouth mason jars. A 50 ml conical tube cap containing 0.56 g of soda lime was added to the bottom of each jar. The pollen mix was contained inside an aluminum pan (45 mm×15 mm) and stacked above the soda lime container separated by a piece of metal 841 micrometer mesh screen. An iButtonLink DS1923-F5 # Hygrochron temperature and humidity data logger was stacked above the pollen mix container to monitor internal relative humidity of the seven 125 ml pollen storage vessels. The iButtonLink DS1923-F5 # Hygrochron temperature and humidity data logger was separated from the pollen mix container using a piece of metal 841 micrometer mesh screen. Regular mouth mason jar lids were equipped with a septum for gas analysis and a series of different sized perforations were made in each lid with a handheld die press (i.e., each lid received a different size perforations). The perforation sizes included 0.0, 1.588 mm ( 1/16-inch), 2.381 mm ( 3/32-inch), 3.175 mm (⅛-inch), 4.763 mm ( 3/16-inch), 6.35 mm (¼-inch), and 9.525 mm (⅜-inch) diameters. Disks of Tyvek were cut from a standard FedEx envelope and were taped over the perforations. This series of increasingly large perforations covered in a Tyvek breathable barrier was used to assess the impact of breathable barrier surface area on pollen storage. The series of different perforation sizes provided a range of surface areas from 2.0 mm² to 71.3 mm² (Table 17). The lid without a perforation (0.0 mm in Table 18) was used as a negative control and allowed the pollen mix to respire in the sealed container until anaerobic conditions were obtained and the stored pollen was rendered inviable. The standard mouth mason jar lids were sealed to the 125 ml mason jars with a mason jar lid band.

A positive control was included by adding 4.89 g of pollen mix to a 400 ml GA7 Magenta box containing 0.68 g of added soda lime. Soda lime was contained in a 50 ml conical tube cap placed at the bottom of the GA7 box. 100 ul of water was added to the bottom of the GA7 container. The pollen mix was contained inside a PLA plastic cup (˜45 mm×30 mm) and positioned above the open soda lime container. An iButtonLink DS1923-F5 # Hygrochron temperature and humidity data logger was stacked above the pollen mix container to monitor internal relative humidity of the GA7 Magenta box. The GA7 lid was placed on top of the GA7 box and the gas exchange gap between box and lid was sealed by wrapping a single layer of 25.4 mm (1-inch) wide parafilm around the open gap. The parafilm was stretched as is normal practice in plant tissue culture to create a tight seal. The GA7 gas exchange gap between box and lid surface area is calculated to total 42 mm². All Treatments were stored at 6° C. at 1 atm pressure for 5 days. After 5 days of storage, pollen viability, final PMC relative to initial PMC, and end point gas analysis of O₂ and CO₂ was assessed.

Two replicate pollinations were made onto receptive silks of primary ears on hybrid maize plants for all treatments except for the negative control treatment that had no breathable barrier. The negative control was non-viable after 5 days of storage, vessel pressure had reduced to −22 kPa gauge due to pollen respiration, had an internal O₂ content of 0.4%, an internal CO₂ content of 0.1%, and a final relative PMC of 103.2%. Pollen viability was 64% to 78% after 5 days of storage for the positive control and treatments with Tyvek covered perforations ranging between 1.588 mm ( 1/16-inch) and 9.525 mm (⅜-inch). Treatments with Tyvek covered perforations ranging between 1.588 mm ( 1/16-inch) and 9.525 mm (⅜-inch) all had a higher rate of pollen tube germination than the GA7 Magenta box and parafilm with added soda lime positive control. Relative final PMCs ranged from 97.3% to 108.2% after 5 days of storage for the positive control and treatments with Tyvek covered perforations ranging between 1.588 mm ( 1/16-inch) and 9.525 mm (⅜-inch). The positive control had the lowest final PMC. Relative final PMC appeared to decrease as the Tyvek breathable barrier surface area increased. An internal gas sample was acquired from each treatment and indicated an O₂ content ranging between 19.1% and 19.5% and a CO₂ content ranging between 0.1% and 0.3%. Treatments with higher Tyvek breathable barrier surface area and the positive control had lower O₂ contents and higher CO₂ contents than treatments with lower Tyvek breathable barrier surface areas. The positive control and all Tyvek treatments provided sufficient gas exchange while minimizing water vapor transmission at all surface areas tested. The presence of soda lime provided sufficient sequestration of CO₂ in conjunction with both types of breathable barrier at the tested surface areas. The iButtonLink DS1923-F5 # Hygrochron temperature and humidity data logger indicated a saturated rH above 98% was obtained throughout the duration of storage inside storage vessels for all treatments. Seed set was observed to increase as the surface area of the Tyvek breathable barrier increased compared to the positive control. Average seed sets ranged from 686 kernels/ear to 788 kernels/ear, indicating that pollen was viable after 5 days of storage.

TABLE 17
Perforation Diameter to Surface Area and
Area of Tyvek per gram of pure pollen

| Perforation | Tyvek surface | Gram pollen | Tyvek surface area per |
|---|---|---|---|
| Diameter (mm) | area (mm²) | (g) | gram pollen (mm²) |
| 1.588 | 2.0 | 2.67 | 0.74 |
| 2.381 | 4.5 | 2.67 | 1.67 |
| 3.175 | 7.9 | 2.67 | 2.97 |
| 4.763 | 17.8 | 2.67 | 6.67 |
| 6.350 | 31.7 | 2.67 | 11.86 |
| 9.525 | 71.3 | 2.67 | 26.69 |

TABLE 18
Pollen viability assessment and gas composition analysis

| | | Breathable | | Final PMC | | |
|---|---|---|---|---|---|---|
| | Breathable | Barrier | Pollen Tube | Relative | % O₂ | % CO₂ |
| Days | Barrier | Surface Area | Gemination | to Initial | at 1 atm, | at 1 atm, |
| Stored | Treatment | (mm²) | (%) | PMC (%) | 22° C. | 22° C. |
| 0 | Fresh | NA | 66 | NA | NA | NA |
| | Pollen:Silica | | | | | |
| | Mix | | | | | |
| 5 | Sealed Vessel | 0.0 | 0 | 103.2 | 0.4 | 0.1 |
| 5 | Tyvek | 2.0 | 72 | 106.7 | 19.5 | 0.1 |
| 5 | Tyvek | 4.5 | 78 | 106.0 | 19.5 | 0.1 |
| 5 | Tyvek | 7.9 | 70 | 105.9 | 19.4 | 0.2 |
| 5 | Tyvek | 17.8 | 70 | 108.2 | 19.4 | 0.2 |
| 5 | Tyvek | 31.7 | 69 | 101.7 | 19.4 | 0.3 |
| 5 | Tyvek | 71.3 | 72 | 101.5 | 19.2 | 0.3 |
| 5 | Parafilm | 42.0 | 64 | 97.3 | 19.1 | 0.2 |

NA = Not Applicable

TABLE 19
Average seed set

| | | Breathable | | | |
|---|---|---|---|---|---|
| | | Barrier | | | |
| | Breathable | Surface | | | |
| Days | Barrier | Area | Replicate | Avg Seed | |
| Stored | Treatment | (mm²) | Pollinations | Set/Ear (k) | Stdev (k) |
| 5 | Tyvek | 2.0 | 2 | 686 | 24 |
| 5 | Tyvek | 4.5 | 2 | 733 | 0 |
| 5 | Tyvek | 7.9 | 2 | 764 | 16 |
| 5 | Tyvek | 17.8 | 2 | 767 | 3 |
| 5 | Tyvek | 31.7 | 2 | 787 | 15 |
| 5 | Tyvek | 71.3 | 2 | 788 | 36 |
| 5 | Parafilm | 42.0 | 2 | 698 | 36 |

### Experiment 3: Surface Area Factorial for Perforations in an Otherwise Airtight Container (i.e., Mason Jar and Lid) with Added Soda Lime

For the following experiment, 24.832 g of pollen was mixed with 12.416 g of 10 μm crystalline silica (2:1 pollen:silica by weight) for a total of 37.248 g pollen mix with an initial fresh mix PMC of 35.41%. A total of 4.0 g pollen mix (2.67 g pollen) was added to each of six 125 ml regular mouth mason jars. A 50 ml conical tube cap containing 0.56 g of soda lime was added to the bottom of each mason jar. The pollen mix was contained inside an aluminum pan (45 mm×15 mm). The pans containing pollen were stacked above the soda lime container and separated by a piece of metal 841 micrometer mesh screen. An iButtonLink DS1923-F5 # Hygrochron temperature and humidity data logger was stacked above the pollen mix container to monitor internal relative humidity of the six 125 ml pollen storage vessels. The iButtonLink DS1923-F5 # Hygrochron temperature and humidity data logger was separated from the pollen mix container using a piece of metal 841 micrometer mesh screen. Regular mouth mason jar lids equipped with a septum for gas analysis were placed on top of each 125 ml mason jar and sealed with a standard mason jar lid band.

Using a handheld die press, one mason jar lid was modified to include a 1.588 mm perforation ( 1/16-inch) and a second mason jar lid was modified to include a 3.175 mm (⅛-inch) perforation. A disk of Tyvek was cut from a standard FedEx envelope was taped over each of the two perforations to act as a breathable barrier. An impermeable plate seal film was applied to the outer perimeter of the Tyvek to seal the Tyvek to the container lid. The two Tyvek breathable barrier treatments were used as positive controls. Each of the four remaining 125 ml pollen storage vessels received different sized perforations in the lids. Perforations were created using various gauge sizes of needles (18 gauge, 20 gauge, 23 gauge, and 30 gauge needles). The needles provided an open micro-perforation from the interior of the pollen storage vessels to the outside atmosphere and a range of open surface areas from 0.015 mm² to 0.552 mm². The 125 ml mason jar pollen storage vessels were sealed with a mason jar lid band. All treatments were stored at 6° C. at 1 atm pressure for 5 days. After 5 days of storage, pollen viability, final PMC relative to initial PMC, and end point gas analysis of O₂ and CO₂ was assessed.

Two replicate pollinations were made onto receptive silks of primary ears on hybrid maize plants for all treatments except the 30 gauge micro-perforation treatment. The 30 gauge treatment was non-viable after 5 days of storage, had an anaerobic internal O₂ content of 0.2%, a CO₂ content of 0.1%, and a final relative PMC of 98.5%. Pollen tube germination was 48% for the 3.175 mm perforation (⅛-inch) and 50% for the 1.588 mm ( 1/16-inch) perforation positive control Tyvek breathable barrier treatments. Pollen tube germination ranged between 61% to 62% after 5 days of storage for the 18 gauge, 20 gauge, and 23 gauge treatments. The open micro-perforation treatments demonstrated higher pollen tube germination compared to the Tyvek treatments. Relative final PMCs ranged from 94.4% to 103.0% after 5 days of storage for all treatments. An internal gas sample was acquired from each Tyvek treatment and indicated that O₂ content ranged between 18.5% and 18.8%, while CO₂ contents ranged between 0.1% and 0.2%. The two Tyvek breathable barrier treatments provided sufficient gas exchange while minimizing water vapor transmission at all surface areas tested. The open micro-perforations reduced and constrained gas exchange. An internal gas sample measured O₂ contents of 8.2%, 2.2%, and 6.1% from the 18 gauge, 20 gauge, and 23 gauge open micro-perforation treatments, respectively. A CO₂ content of 0.2% was measured for all three treatments. The presence of soda lime provided sufficient sequestration of CO₂. The iButtonLink DS1923-F5 # Hygrochron temperature and humidity data logger indicated a saturated rH above 98% was obtained throughout the duration of storage inside storage vessels for all treatments. Seed set was observed to increase as the surface area of Tyvek breathable barrier increased. Seed set was observed to increase as the open area of micro-perforations increased from 23 gauge to 18 gauge. The Tyvek breathable barrier treatments had greater seed sets than any open micro-perforation treatment. An average seed set ranging between 629 kernels/ear and 770 kernels/ear indicate the pollen was viable after 5 days of storage.

TABLE 20
Perforation Diameter to Surface Area and
Perforation Area per gram of pure pollen.
All treatments used 2.67 g pure pollen per container.

| | Perforation | Surface area | Area of perforation mm² |
|---|---|---|---|
| Needle Gauge | Diameter (mm) | (mm²) | per gram pure pollen |
| 30 | 0.14 | 0.015 | 0.110 |
| 23 | 0.318 | 0.079 | 0.250 |
| 20 | 0.584 | 0.268 | 0.459 |
| 18 | 0.838 | 0.552 | 0.658 |

TABLE 21
Perforation Diameter to Surface Area and
Area of Tyvek per gram of pure pollen.
All treatments used 2.67 g pure pollen per container.

| Perforation | Surface area | Tyvek surface area per |
|---|---|---|
| Diameter (mm) | (mm²) | gram pollen (mm²) |
| 1.588 | 2.0 | 0.74 |
| 3.175 | 7.9 | 2.97 |

TABLE 22
Pollen viability assessment and gas composition analysis

| | Breathable | | | | |
|---|---|---|---|---|---|
| | Barrier | Pollen | Final PMC | % O₂ | % CO₂ |
| | Surface | Tube | Relative to | at 1 | at 1 |
| Days | Area | Gemination | Initial PMC | atm, | atm, |
| Stored | (mm²) | (%) | (%) | 22° C. | 22° C. |
| 0 | Fresh | 53 | NA | NA | NA |
| | Pollen:Silica | | | | |
| | Mix | | | | |
| 5 | 0.015 | 0 | 98.5 | 0.2 | 0.1 |
| 5 | 0.079 | 61 | 98.1 | 6.1 | 0.2 |
| 5 | 0.268 | 61 | 94.4 | 2.2 | 0.2 |
| 5 | 0.552 | 62 | 103 | 8.2 | 0.2 |
| 5 | 2.0 | 50 | 101.1 | 18.5 | 0.2 |
| 5 | 7.9 | 48 | 94.5 | 18.8 | 0.1 |

NA = Not Applicable

TABLE 23
Average seed set

| Days | Breathable Barrier | Replicate | Avg Seed | |
|---|---|---|---|---|
| Stored | Surface Area (mm²) | Pollinations | Set/Ear (k) | Stdev (k) |
| 5 | 0.015 | 2 | 0 | 0 |
| 5 | 0.079 | 2 | 668 | 82 |
| 5 | 0.268 | 2 | 630 | 21 |
| 5 | 0.552 | 2 | 687 | 16 |
| 5 | 2.0 | 2 | 768 | 15 |
| 5 | 7.9 | 2 | 770 | 28 |

### Experiment 4: 3M Micropore Tape as an Alternative Breathable Barrier with Soda Lime (5 Day)

For micropore tape evaluation, 10.396 g of pollen was mixed with 5.190 g of 10 μm crystalline silica (2:1 pollen:silica by weight) for a total of 15.5856 g pollen mix with an initial fresh mix PMC of 34.90%. A total of 4.0 g pollen mix (2.67 g pollen) was added to three 125 ml regular mouth mason jars and a 50 ml conical tube cap containing 0.56 g of soda lime was added to the bottom of each mason jar. The pollen mix was contained inside an aluminum pan (˜45 mm×15 mm). The pan containing the pollen mix was stacked above the soda lime container and separated by a piece of metal 841 micrometer mesh screen. An iButtonLink DS51923-F5 # Hygrochron temperature and humidity data logger was stacked above the pollen mix container to monitor internal relative humidity in each of the three 125 ml pollen storage vessels. The iButtonLink DS1923-F5 # Hygrochron temperature and humidity data logger was separated from the pollen mix container using a piece of metal 841 micrometer mesh screen. Regular mouth mason jar lids were equipped with a septum for gas analysis. Of the three mason jar lids used, one lid was modified to include a 1.588 mm ( 1/16-inch) perforation and two lids were modified to include a 3.175 mm (⅛-inch) perforation using a handheld die press. A disk of Tyvek was cut from a standard FedEx envelope and was taped over the 3.175 mm (⅛-inch) perforation in one of the three lids. An impermeable plate seal film was applied to the outer perimeter of the Tyvek to seal the Tyvek to the container lid. The 3.175 mm (⅛-inch) perforation with the Tyvek breathable barrier was used as a positive control. 3M brand micropore tape was placed over the 1.588 mm ( 1/16-inch) perforation in the second lid and the same micropore tape was placed over the 3.175 mm (⅛-inch) perforation in the third lid. The lids were sealed onto the 125 ml mason jar pollen storage vessels using mason jar lid bands. All treatments were stored at 6° C. at 1 atm pressure for 5 days. After 5 days of storage, pollen viability, final PMC relative to initial PMC, and end point gas analysis of O₂ and CO₂ was assessed.

A single pollination was made onto receptive silks of primary ears on hybrid maize plants for each treatment. Pollen viability qualitatively scored a ranking of 4 (>41 to 60% germination rate; see Table 29) after 5 days of storage for all treatments. The relative PMCs ranged from 103.8% to 106.0% across treatments. An internal gas sample was acquired from each jar and 02 contents ranged from 18.4% to 19.4% while CO₂ contents ranged from 0.1% to 0.2%. The 1.588 mm ( 1/16-inch) micropore tape treatment indicated the lowest O₂ content of 18.4% while also indicating a CO₂ content of 0.2%. Both the Tyvek and micropore tape breathable barriers provided sufficient gas exchange while minimizing water vapor transmission at the surface areas tested. The presence of soda lime provided sufficient sequestration of CO₂ in conjunction to the breathable barriers at the tested surface areas. The iButtonLink DS1923-F5 # Hygrochron temperature and humidity data logger indicated a saturated rH above 98% was obtained throughout the duration of storage inside the storage vessel for all treatments. Both the 3.175 mm (⅛-inch) Tyvek breathable barrier treatment and the 1.588 mm ( 1/16-inch) micropore tape breathable barrier treatment reached 100% saturated rH, while the 3.175 mm (⅛-inch) micropore tape breathable barrier treatment only reached 98% rH. Seed sets of 711 kernels/ear, 751 kernels/ear, and 696 kernels/ear were obtained for the 3.175 mm (⅛-inch) Tyvek breathable barrier, the 1.588 mm ( 1/16-inch) micropore tape breathable barrier, and the 3.175 mm (⅛-inch) micropore tape breathable barrier, respectively, indicating that the pollen was viable after 5 days of storage.

TABLE 24
Pollen viability assessment, gas composition analysis, seed set (k)

| | Breathable | | Final PMC | | | |
|---|---|---|---|---|---|---|
| | Barrier | Breathable | Relative | % O₂ | % CO₂ | |
| Days | Surface Area | Barrier | to Initial | at 1 atm, | at 1 atm, | Seed |
| Stored | (mm²) | Treatment | PMC (%) | 22° C. | 22° C. | Set (k) |
| 5 | 2.0 | Micropore | 105.7 | 18.4 | 0.2 | 751 |
| | | Tape | | | | |
| 5 | 7.9 | Tyvek | 106.0 | 19.4 | 0.1 | 711 |
| 5 | 7.9 | Micropore | 103.8 | 19.2 | 0.1 | 696 |
| | | Tape | | | | |

### Experiment 5: Surface Area Factorial with Tyvek Versus Micropore Tape with Soda Lime (5 Day)

For further evaluation, 10.89 g of pollen was mixed with 5.445 g of 10 μm crystalline silica (2:1 pollen:silica by weight) of for a total of 16.335 g pollen mix with an initial fresh mix PMC of 33.77%. A total of 2.0 g pollen mix (1.335 g pollen) was added to eight 125 ml regular mouth mason jars. A 50 ml conical tube cap containing 0.28 g of soda lime was added to the bottom of each mason jar. The pollen mix was contained inside an aluminum pan (45 mm×15 mm). The pan containing the pollen mix was stacked above the soda lime container and separated by a piece of metal 841 micrometer mesh screen. An iButtonLink DS1923-F5 # Hygrochron temperature and humidity data logger was stacked above the pollen mix in each container to monitor internal relative humidity of the eight 125 ml pollen storage vessels. The iButtonLink DS1923-F5 # Hygrochron temperature and humidity data logger was separated from the pollen mix container using a piece of metal 841 micrometer screen. Regular mouth mason jar lids were equipped with a septum for gas analysis. Eight total mason jar lids were modified to include one perforation per lid using handheld die press. Two lids each were modified to include a 1.588 mm, ( 1/16-inch), 3.175 mm (⅛-inch), 6.35 mm (¼-inch), and a 9.525 mm (⅜-inch) perforation. Disks of Tyvek were cut from a standard FedEx envelope and were taped over one lid at each preformation size. An impermeable plate seal film was applied to the outer perimeter of the Tyvek to seal the Tyvek to the lid. A piece of 3M Micropore Tape was applied over the second lid at each perforation size. In addition to evaluating the micropore tape against Tyvek, this experiment also assessed the impact of increasing or decreasing breathable barrier surface area. Perforated lids were sealed to each 125 ml mason jar pollen storage vessels using mason jar lid bands. All treatments were stored at 6° C. at 1 atm pressure for 5 days. After 5 days of storage, pollen viability, final PMC relative to initial PMC, and end point gas analysis of O₂ and CO₂ was assessed.

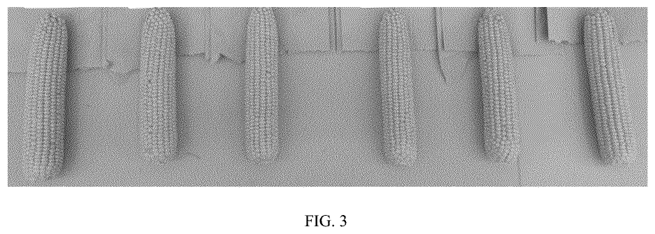

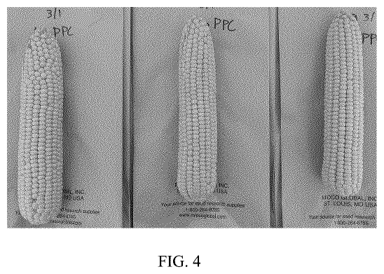

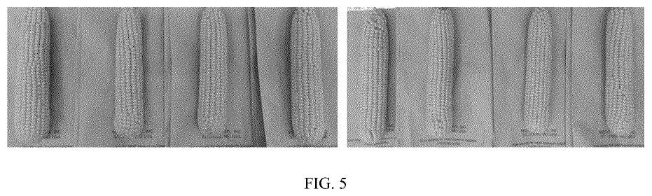

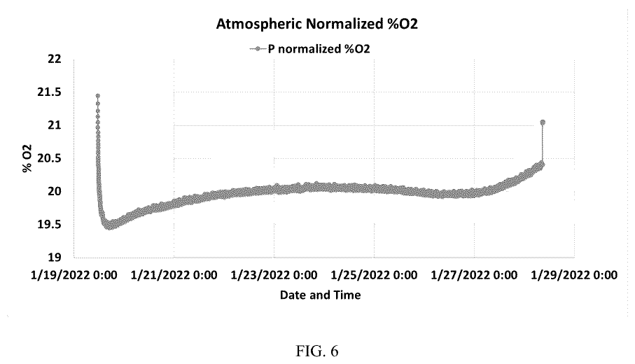

A single replicate pollination was made onto receptive silks of primary ears on hybrid maize plants for each treatment. Pollen tube germination ranged between 55% to 61% for the Tyvek treatments and between 47% to 62% for the micropore tape treatments after 5 days of storage. An internal gas sample was acquired from each treatment. O₂ contents ranged from 19.5% to 19.9% across the Tyvek treatments while CO₂ contents all measured 0.1%. O₂ contents ranged from 19.3% to 19.9% across the micropore tape treatments while CO₂ contents all measured 0.1%. O₂ contents increased as the surface area of both types of breathable barriers increased. The presence of soda lime provided sufficient sequestration of CO₂ in conjunction with both types of breathable barriers at the surface areas tested. The relative final PMCs ranged from 102.2% to 84.3% across the Tyvek treatments and 101.5% to 78.6% for the micropore tape treatments. The relative final PMCs were observed to decrease as the surface area of both types of breathable barriers increased. Both the Tyvek and micropore tape covered 9.525 mm (⅜-inch) perforation treatments had high pollen moisture loss. In general, the Tyvek breathable barrier treatments retained higher pollen moisture contents compared to the micropore tape treatments. The iButtonLink DS1923-F5 # Hygrochron temperature and humidity data loggers indicated a saturated rH above 98% was obtained throughout the duration of storage for the both the Tyvek and micropore tape 1.588 mm ( 1/16-inch) and 3.175 mm (⅛-inch) perforation treatments. The iButtonLink DS1923-F5 # Hygrochron temperature and humidity data loggers indicated a rH above 96% was obtained throughout the duration of storage for the Tyvek covered 6.35 mm (¼-inch) and 9.525 mm perforations and the 6.35 mm (¼-inch) micropore tape covered perforation treatments. A rH higher than 94% was observed throughout the duration of storage for the 9.525 mm (⅜-inch) micropore tape covered perforation treatment. Seed sets exceeding 500 kernels/ear for all eight treatments indicated that the pollen was viable (FIG. 3).

TABLE 25
Hole Diameter to Surface Area and Area of barrier per gram of pure
pollen. All treatments used 1.335 grams of pollen per container.

| Perforation | Perforation | Breathable barrier surface |
|---|---|---|
| Diameter | Surface area | area per gram of pollen |
| (mm) | (mm²) | (mm²) |
| 1.588 | 2.0 | 1.48 |
| 3.175 | 7.9 | 5.93 |
| 6.350 | 31.7 | 23.72 |
| 9.525 | 71.3 | 53.38 |

TABLE 26
Pollen viability assessment and gas composition analysis

| | | Breathable | Pollen | Final PMC | | |
|---|---|---|---|---|---|---|
| | Breathable | Barrier | Tube | Relative | % O₂ | % CO₂ |
| Days | Barrier | Surface Area | Gemination | to Initial | at 1 atm, | at 1 atm, |
| Stored | Treatment | (mm²) | (%) | PMC (%) | 22° C. | 22° C. |
| 0 | Fresh | NA | 62 | NA | NA | NA |
| | Pollen:Silica | | | | | |
| | Mix | | | | | |
| 5 | Tyvek | 2.0 | 55 | 102.2 | 19.5 | 0.1 |
| 5 | Tyvek | 7.9 | 58 | 97.0 | 19.7 | 0.1 |
| 5 | Tyvek | 31.7 | 61 | 98.3 | 19.6 | 0.1 |
| 5 | Tyvek | 71.3 | 55 | 84.3 | 19.9 | 0.1 |
| 5 | Micropore Tape | 2.0 | 51 | 97.8 | 19.3 | 0.1 |
| 5 | Micropore Tape | 7.9 | 55 | 101.5 | 19.6 | 0.1 |
| 5 | Micropore Tape | 31.7 | 62 | 93.8 | 19.7 | 0.1 |
| 5 | Micropore Tape | 71.3 | 47 | 78.6 | 19.9 | 0.1 |

NA = Not Applicable

TABLE 27
Average seed set

| | | Breathable | |
|---|---|---|---|
| Days | Breathable | Barrier Surface | Seed |
| Stored | Barrier Treatment | Area (mm²) | Set/Ear |
| 5 | Tyvek | 2.0 | 690 |
| 5 | Tyvek | 7.9 | 727 |
| 5 | Tyvek | 31.7 | 714 |
| 5 | Tyvek | 71.3 | 621 |
| 5 | Micropore Tape | 2.0 | 641 |
| 5 | Micropore Tape | 7.9 | 604 |
| 5 | Micropore Tape | 31.7 | 576 |
| 5 | Micropore Tape | 71.3 | 624 |

### Experiment 6: Surface Area Factorial with Perforations in an Otherwise Airtight Container (i.e., Mason Jar and Lid) and Soda Lime (5 Day)

For experiment 6, 6.48 g of pollen was mixed with 3.24 g of 10 μm crystalline silica (2:1 pollen:silica by weight) of for a total of 9.72 g pollen mix with an initial fresh mix PMC of 35.78%. A total of 1.95 g pollen mix (1.3007 g pollen) was added to four 125 ml regular mouth mason jars and 1.6 g pollen mix (1.0672 g pollen) was added to a fifth 125 ml regular mouth mason jar. A 50 ml conical tube cap containing 0.28 g of soda lime was added to the bottom of each container. The pollen mix was contained inside an aluminum pan (45 mm×15 mm). The pan containing pollen mix was stacked above the soda lime container and separated by a piece of metal 841 micrometer mesh screen. An iButtonLink DS1923-F5 # Hygrochron temperature and humidity data logger was stacked above the pollen mix container to monitor internal relative humidity in each of the eight 125 ml pollen storage vessels. The iButtonLink DS1923-F5 # Hygrochron temperature and humidity data logger was separated from the pollen mix pan using a piece of metal 841 micrometer mesh screen. Five regular mouth mason jar lids were equipped with a septum for gas analysis. One lid was modified into include a 3.175 mm perforation (⅛-inch) made with a handheld die press. A disk of Tyvek was cut from a standard FedEx envelope and was taped over the 3.175 mm (⅛-inch) perforation. An impermeable plate seal film was applied to the outer perimeter of the Tyvek to seal the Tyvek to the container lid. The 3.175 mm perforation (⅛-inch) Tyvek breathable barrier jar received 1.95 g pollen mix and was used as a positive control in the experiment.

The remaining four lids were modified to include perforations using a handheld die press. One lid each was modified to include a 1.588 mm ( 1/16-inch), 3.175 mm (⅛-inch), 6.35 mm (¼-inch), or a 9.525 mm (⅜-inch) diameter perforation. Perforations in each of the four lids were left open to atmosphere during storage to determine if the perforation could act as a breathable barrier without the addition of a covering. The experiment also assessed the impact open perforation surface area. The 1.588 mm ( 1/16-inch), 3.175 mm (⅛-inch), and 6.35 mm (¼-inch) open perforation treatments each received 1.95 g of pollen mix while the 9.525 mm (⅜-inch) open perforation treatment received 1.6 g of pollen mix. Each lid was sealed onto a 125 ml mason jar using mason jar lid bands. All treatments were stored at 6° C. at 1 atm pressure for 5 days. After 5 days of storage, pollen viability, final PMC relative to initial PMC, and end point gas analysis of O₂ and CO₂ was assessed.

A single pollination was made onto receptive silks of primary ears on hybrid maize plants for each treatment. Pollen tube germination was between 50% and 55% for all treatments except for the 9.525 mm (⅜-inch) open perforation treatment after 5 days of storage. The 9.525 mm (⅜-inch) open perforation treatment was originally qualitatively scored a ranking of 2 (<10% germination rate; see Table 29) after 60 minutes of pollen tube germination. A lag in pollen tube germination was observed and the sample was incubated for an additional five hours at 100% rH. The 9.525 mm (⅜-inch) open perforation treatment demonstrated 43% pollen tube germination after five hours. An internal gas sample was acquired for each treatment and O₂ contents ranged from 19.9% to 20.0%, while all CO₂ contents measured 0.1%. The presence of soda lime provided sufficient sequestration of CO₂ in conjunction with the Tyvek and open perforation breathable barriers. The relative final PMC of the 3.175 mm (⅛-inch) Tyvek covered perforation positive control was 91.7%. The internal rH inside the 6° C. storage chamber fluctuated between 30% and 50% during the duration of storage. The relative final PMCs ranged from 102.2% to 84.3% across the Tyvek treatments and 94.9% to 46.0% for the series of open perforation treatments. The relative final PMCs decreased as the surface area of the open perforations increased. The 6.35 mm (¼-inch) and 9.525 mm (⅜-inch) open perforation treatments had large pollen moisture losses. The iButtonLink DS1923-F5 # Hygrochron temperature and humidity data logger indicated a saturated rH above 98% was obtained throughout the duration of storage for the 3.175 mm (⅛-inch) Tyvek covered perforation positive control treatment and for both the 1.588 mm ( 1/16-inch) and 3.175 mm open perforation (⅛-inch) treatments. The iButtonLink DS1923-F5 # Hygrochron temperature and humidity data logger data indicated a rH above 92% was obtained throughout the duration of storage for the 6.35 mm (¼-inch) open perforation treatment. A rH of greater than 85% was observed throughout the duration of storage for the 9.525 mm (⅜-inch) open perforation treatment. Seed sets exceeding 700 kernels/ear were obtained for the 3.175 mm (⅛-inch) Tyvek, 1.588 mm ( 1/16-inch), 3.175 mm (⅛-inch), and 6.35 mm (¼-inch) open perforation treatments, indicating that the pollen was viable after 5 days of storage. The 9.525 mm (⅜-inch) open perforation treatment produced 322 kernels/ear, suggesting that the larger moisture loss from the pollen during storage negatively impacted seed set.

TABLE 28
Pollen viability assessment and gas composition analysis

| | | Breathable | Pollen | Final PMC | | |
|---|---|---|---|---|---|---|
| | Breathable | Barrier | Tube | Relative to | % O₂ | % CO₂ |
| Days | Barrier | Surface Area | Gemination | Initial | at 1 atm, | at 1 atm, |
| Stored | Treatment | (mm²) | (%) | PMC (%) | 22° C. | 22° C. |
| 0 | Fresh | NA | 67 | NA | NA | NA |
| | Pollen:Silica | | | | | |
| | Mix | | | | | |
| 5 | Tyvek | 5.93 | 55 | 91.7 | 19.9 | 0.1 |
| 5 | Open | 1.48 | 50 | 94.9 | 19.9 | 0.1 |
| 5 | Open | 5.93 | 54 | 87.2 | 19.9 | 0.1 |
| 5 | Open | 23.72 | 51 | 71.4 | 19.9 | 0.1 |
| 5 | Open | 53.38 | 43 | 46.0 | 20 | 0.1 |

NA = Not Applicable

TABLE 29
Average seed set

| | Breathable | Breathable | |
|---|---|---|---|
| Days | Barrier | Barrier Surface | Seed Set |
| Stored | Treatment | Area (mm²) | (k) |
| 5 | Tyvek | 5.93 | 754 |
| 5 | Open | 1.48 | 713 |
| 5 | Open | 5.93 | 727 |
| 5 | Open | 23.72 | 761 |
| 5 | Open | 53.38 | 322 |

TABLE 30
The ranking scale for qualitative pollen
tube germination used in the experiments.

| Scale | Germination Rate |
|---|---|
| 5 | >60% |
| 4 | 41-60% |
| 3 | 21-40% |
| 2 | 1-20% |
| 1 | 0% |

N = 4 for all pollen tube germination assessments in the examples above.

### Experiment 7: Larger Perforations with Tyvek & Micropore Tape Breathable Barriers with Soda Lime (5 Day)

To evaluate larger perforations, 11.8924 g of pollen was mixed with 5.946 g of 10 μm crystalline silica for a total of 17.839 g pollen mix with an initial fresh mix PMC of 35.9%. Pollen was mixed at a ratio of two parts pollen, one part carrier by weight. A total of 2.0 g pollen mix (1.335 g pollen) was added to six 125 ml regular mouth mason jars. A 50 ml conical tube cap containing 0.28 g of soda lime was added to the bottom of each mason jar. The pollen mix was contained inside an aluminum pan (45 mm×15 mm). The pollen mix container was stacked above the soda lime container and separated by a piece of metal 841 micrometer mesh screen. An iButtonLink DS1923-F5 # Hygrochron temperature and humidity data logger was stacked above the pollen mix container to monitor internal relative humidity in each of the six 125 ml pollen storage vessels and was separated from the pollen mix container using a piece of metal 841 micrometer mesh screen. Regular mouth mason jar lids were equipped with a septum for gas analysis.

Six lids were modified to include perforations using a handheld die press. Two lids each were modified to include a 3.175 mm (⅛-inch), 11.113 mm ( 7/16-inch), and a 12.7 mm (½-inch) perforation. Disks of Tyvek were cut from a standard FedEx envelope and were taped over one example of each perforation size for a total of three Tyvek treatments. An impermeable plate seal film was applied to the outer perimeter of the Tyvek to seal the Tyvek to the container lid. A piece of 3M micropore tape was applied over the second set of lids, one at each of the three perforation sizes. In addition to evaluating micropore tape compared to Tyvek breathable barriers, this experiment also assessed the impact of increasing or decreasing surface area for both breathable barriers. Lids were sealed to the six 125 ml mason jar pollen storage vessels using mason jar lid bands. All treatments were stored at 6° C. at 1 atm pressure for 5 days. After 5 days of storage, pollen viability and final PMC relative to initial was assessed.

A single pollination was made onto receptive silks of primary ears on hybrid maize plants for each treatment. Pollen tube germination was between 53% and 55% for Tyvek treatments and 47% to 50% for the micropore tape treatments after 5 days of storage. The relative final PMCs ranged from 101.5% to 98.0% across the Tyvek treatments and 94.2% to 83.3% for the micropore tape treatments. The relative final PMCs were observed to decrease as the surface area of both breathable barrier types increased. The Tyvek treatments maintained higher pollen moisture contents than the equivalent micropore tape treatments. The iButtonLink DS1923-F5 # Hygrochron temperature and humidity data logger indicated a saturated rH above 98% was obtained throughout the duration of storage for the both the Tyvek and Micropore Tape covered 3.175 mm perforation (⅛-inch) treatments. The iButtonLink DS1923-F5 # Hygrochron temperature and humidity data indicated a rH above 96% was obtained throughout the duration of storage for the Tyvek covered 11,113 mm ( 7/16-inch) ( 7/16-inch) and 12.7 mm (½-inch) perforation treatments. The iButtonLink DS1923-F5 # Hygrochron temperature and humidity data indicated an approximate 36-hour lag to obtain a rH above 98% for the micropore tape covered 11.113 mm ( 7/16-inch) and 12.7 mm (½-inch) perforation treatments. The micropore tape covered 11.113 mm ( 7/16-inch) and 12.7 mm (½-inch) perforation treatments had relative final PMCs of 83.3% and 90.1%, respectively, indicating that for Micropore Tape, these surface areas per gram of pollen were too large to maintain a sufficient pollen moisture content. A seed set range of 614 kernels/ear to 755 kernels/ear indicated the pollen was viable after 5 days of storage.

TABLE 31
Perforation Diameter to Surface Area
and Area of barrier per gram of pure pollen.
All treatments used 1.355 grams of pure pollen.

| Perforation | Perforation | Perforation surface area |
|---|---|---|
| Diameter | surface area | per gram pollen |
| (mm) | (mm²) | (mm²) |
| 3.175 | 7.9 | 5.93 |
| 11.113 | 96.99 | 72.65 |
| 12.700 | 126.68 | 94.89 |

TABLE 32
Pollen viability assessment and Seed Set

| | | | | Final | |
|---|---|---|---|---|---|
| | | Breathable | Pollen | PMC | |
| | | Barrier | Tube | Relative | |
| | Breathable | Surface | Gemi- | to Initial | |
| Days | Barrier | Area | nation | PMC | Seed |
| Stored | Treatment | (mm²) | (%) | (%) | Set (k) |
| 0 | Fresh | NA | 57 | NA | ND |
| | Pollen:Silica | | | | |
| | Mix | | | | |
| 5 | Tyvek | 7.9 | 55 | 100.5 | 660 |
| 5 | Tyvek | 96.99 | 55 | 99.0 | 614 |
| 5 | Tyvek | 126.68 | 53 | 98.0 | 687 |
| 5 | Micropore Tape | 7.9 | 50 | 94.2 | 755 |
| 5 | Micropore Tape | 96.99 | 50 | 83.3 | 734 |
| 5 | Micropore Tape | 126.68 | 47 | 90.1 | 729 |

NA = Not Applicable;
ND = No Data

### Experiment 8: Larger Pollen Quantity, Direct Stacking in Tube, and Micropore Tape—No Soda Lime (5 Day)

In this experiment, 20.19 g of pollen was mixed with 10.096 g of 10 μm crystalline silica, for a total of 30.286 g pollen mix with an initial fresh mix PMC of 36.10%. Pollen was mixed at a ratio of two parts pollen, one part carrier by weight. A total of 29.675 g pollen mix (˜40 ml) was added directly into a 50 ml conical tube. A 50 ml conical tube cap was modified to include a 9.525 mm (⅜-inch) diameter perforation drilled into the center of the cap. The perforation was covered with 3M micropore tape to provide a breathable barrier surface area of 71.3 mm². The modified cap was placed on the 50 ml conical tube full of pollen maxed and tightened down to seal with the tube. The capped conical tube was stored in a vertical orientation (cap facing directly up) at 6° C. at 1 atm pressure for 5 days. After 5 days of storage, pollen viability and final PMC relative to initial was assessed.

The pollen mix was subsampled at 1.5 cm (Top), 4.0 cm (Middle), and 6.5 cm (Bottom) depths below the surface of the pollen mix. A single pollination was made onto receptive silks of primary ears on hybrid maize plants for each depth. Pollen tube germination was 57% at 1.5 cm, 57% at 4.0 cm, and 59% at 6.5 cm. The relative final PMCs were 98.4% at 1.5 cm, 102.7% at 4.0 cm, and 103.0% at 6.5 cm. The relative final PMCs increased at greater depths from the surface of the pollen mix. The pollen tube germination assessments indicated that the pollen mix can be stacked directly in a storage container up to 7 cm deep with 10 ml of headspace without issue. The relative final PMCs and pollen viability assessments indicated a of micropore tape breathable barrier surface area of 71.3 mm² provided sufficient gas exchange to support pollen respiration while minimizing water vapor transmission from the storage container. A seed set of 773 kernels/ear was obtained using pollen subsampled at the 1.5 cm depth, 749 kernels/ear at 4.0 cm, and 689 kernels/ear at 6.5 cm, indicating that the pollen was viable after 5 days of storage.

TABLE 33
Pollen viability assessment and Seed Set

| | | | | Final | |
|---|---|---|---|---|---|
| | | Depth of | | PMC | |
| | | pollen | | Relative | |
| | | subsample | Pollen | to | |
| | Pollen | in storage | Tube | Initial | Seed |
| Days | subsample | vessel | Gemination | PMC | Set |
| Stored | position | (cm) | (%) | (%) | (k) |
| 0 | Fresh | NA | 51 | NA | ND |
| | Pollen:Silica | | | | |
| | Mix | | | | |
| 5 | Top of pile | 1.5 | 57 | 98.4 | 773 |
| 5 | Middle of pile | 4.0 | 57 | 102.7 | 749 |
| 5 | Base of pile | 6.5 | 59 | 103.0 | 689 |

NA = Not Applicable;
ND = No Data

### Experiment 9: Micropore Tape Surface Area Factorial—No Soda Lime Vs Soda Lime (5 Day)

For experiment 9, 19.43 g of pollen was mixed with 9.72 g of 10 μm crystalline silica for a total of 29.15 g pollen mix with an initial fresh mix PMC of 37.22%. Pollen was mixed at a ratio of two parts pollen, one part carrier by weight. 3.5 g of pollen mix was added to each of eight 125 ml regular mouth mason jars. The pollen mix was contained inside an aluminum pan (45 mm×15 mm). A 50 ml conical tube cap containing 0.49 g of soda lime was added to the bottom of four of the 125 ml regular mouth mason jars. An empty 50 ml conical tube cap was placed at the bottom of the remaining four 125 ml regular mouth mason jars. The pan containing pollen mix was stacked above the 50 ml conical tube cap soda lime container. An iButtonLink DS1923-F5 # Hygrochron temperature and humidity data logger was stacked above the pollen mix container to monitor internal relative humidity in each of the eight 125 ml pollen storage vessels and was separated from the pollen mix container using a piece of metal 841 micrometer mesh screen. Regular mouth mason jar lids were equipped with a septum for gas analysis. Eight total lids were modified to include perforations using a handled die press. Two sets lids each were modified to include a 1.588 mm ( 1/16-inch), a 3.175 mm (⅛-inch), a 6.35 mm (¼-inch), or a 9.525 mm (⅜-inch) perforation. For all eight lids, a piece of 3M Micropore Tape was applied over the perforation. This created two replicate lids each with a micropore tape breathable barrier surface area 2.0 mm², 7.9 mm², 31.7 mm², and 71.3 mm². The purpose of this experiment was to determine if a minimum surface area of micropore tape breathable barrier was required per gram of pollen mix in the absence of soda lime to avoid CO₂ from cellular respiration accumulating to toxic levels. Modified lids were sealed to 125 ml mason jar pollen storage vessels using mason jar lid bands. All treatments were stored at 6° C. at 1 atm pressure for 5 days. After 5 days of storage, pollen viability, final PMC relative to initial PMC, and end point gas analysis of O₂ and CO₂ was assessed.

A single pollination was made onto receptive silks of primary ears on hybrid maize plants for each treatment. Pollen tube germination was between 43% and 56% for all treatments. Treatments with soda lime were observed to have lower percentages of pollen tube germination compared to treatments without soda lime. An internal gas sample was acquired for each treatment and O₂ contents ranged from 19.7% to 19.2%. For treatments with soda lime, the CO₂ contents were all 0.2%. The presence of soda lime provided sufficient sequestration of CO₂ in conjunction with the Micropore Tape breathable barrier at all surface areas tested. The CO₂ contents for treatments without soda lime were 1.2%, 0.6%, 0.4%, and 0.4% for the 2.0 mm², 7.9 mm², 31.7 mm², and 71.3 mm² surface areas tested, respectively. For each surface area tested without the use of soda lime and for the quantity of pollen mix stored, there was sufficient gas exchange to maintain CO₂ accumulation below toxic levels affecting pollen. The relative final PMCs ranged from 100.5% to 87.3% across all 3M micropore tape treatments. No pattern of increase or decrease in relative final PMC was observed regardless of the presence of soda lime. The iButtonLink DS1923-F5 # Hygrochron temperature and humidity data logger indicated a saturated rH above 98% was obtained throughout the duration of storage for all treatments regardless of presence of soda lime. Seed set for single test pollinations from all eight treatments exceeded 500 kernels/ear, indicating that the pollen was viable after 5 days of storage (Table 35). Seed set and pollen tube germination results indicated that the breathable barriers were able to provide sufficient gas exchange to maintain CO₂ below toxic levels without the presence of added soda lime.

TABLE 34
Pollen viability assessment

| | | Breathable | Pollen | Final PMC | | |
|---|---|---|---|---|---|---|
| | Added Soda | Barrier | Tube | Relative to | % O₂ | % CO₂ |
| Days | Lime | Surface Area | Gemination | Initial | at 1 atm, | at 1 atm, |
| Stored | Treatment | (mm²) | (%) | PMC (%) | 22° C. | 22° C. |
| 0 | Fresh | NA | 59 | NA | NA | NA |
| | Pollen:Silica | | | | | |
| | Mix | | | | | |
| 5 | added soda | 1.48 | 47 | 97.2 | 19.2 | 0.2 |
| | lime | | | | | |
| 5 | added soda | 5.93 | 47 | 100.5 | 19.2 | 0.2 |
| | lime | | | | | |
| 5 | added soda | 23.72 | 49 | 92.5 | 19.3 | 0.2 |
| | lime | | | | | |
| 5 | added soda | 53.38 | 43 | 94.3 | 19.4 | 0.2 |
| | lime | | | | | |
| 5 | No soda | 1.48 | 52 | 95.4 | 19.2 | 1.2 |
| | lime | | | | | |
| 5 | No soda | 5.93 | 56 | 96.1 | 19.7 | 0.6 |
| | lime | | | | | |
| 5 | No soda | 23.72 | 50 | 87.3 | 19.7 | 0.4 |
| | lime | | | | | |
| 5 | No soda | 53.38 | 46 | 92.2 | 19.7 | 0.4 |
| | lime | | | | | |

NA = Not Applicable

TABLE 35
Average seed set

| | | Breathable | |
|---|---|---|---|
| | | Barrier | |
| | | Surface | |
| Days | Added Soda Lime | Area | Seed Set |
| Stored | Treatment | (mm²) | (k) |
| 5 | added soda lime | 1.48 | 727 |
| 5 | added soda lime | 5.93 | 720 |
| 5 | added soda lime | 23.72 | 711 |
| 5 | added soda lime | 53.38 | 632 |
| 5 | No soda lime | 1.48 | 792 |
| 5 | No soda lime | 5.93 | 582 |
| 5 | No soda lime | 23.72 | 672 |
| 5 | No soda lime | 53.38 | 694 |

### Experiment 10: Comparison of Single Layer Vs Double Layer Use of Micropore Tape without Soda Lime (5 Day)

The experiment examined the impact of doubling the layers of micropore tape breathable barriers. 27.84 g of pollen was mixed with 13.92 g of 10 μm crystalline silica of for a total of 41.76 g pollen mix with an initial fresh mix PMC of 36.06%. Pollen was mixed at a ratio of two parts pollen, one part carrier by weight. A total of 20.0 g pollen mix was added directly to the bottom of two 125 ml regular mouth mason jars. Regular mouth mason jar lids were equipped with a septum for gas analysis. Two lids were modified to include identical 9.525 mm (⅜-inch) diameter perforations using a handheld die press. A single layer of 3M micropore tape was applied over the perforation on one lid. The perforation on the second lid was covered with two layers of 3M micropore tape, one layer applied on the top of the lid covering the perforation and the second layer applied on the bottom side of the lid covering the perforation. The identical 9.525 mm (⅜-inch) diameter perforations in each lid covered with 3M micropore tape provided a breathable barrier surface area of 71.3 mm². This experimental design tested whether the relative final PMC of pollen could be increased during storage using two layers of micropore tape breathable barrier and to determine if the rate of gas exchange through two layers was sufficient to support pollen respiration compared to a single layer. The treatments were stored at 6° C. at 1 atm pressure for 5 days. At 5 days of storage, pollen viability, final PMC relative to initial PMC, and end point gas analysis of O₂ and CO₂ was assessed.

Three replicate pollinations were made onto receptive silks of primary ears on hybrid maize plants for each treatment. Pollen tube germination was annotated at 55% for the single layer of micropore tape and 56% for the double layer of micropore tape. The relative final PMCs were 102.7% for the single layer and 104.3% for the double layer. This indicates that the relative final PMCs increased when a second layer of micropore tape was added. An internal gas sample was acquired for each of the two storage containers. The O₂ content for the single layer was 19.3% and the CO₂ content was 0.7%. The O₂ content for the double layer was 18.2% and the CO₂ content was 2.2%. The single layer treatment supported greater gas exchange compared to the double layer treatment. A sufficient O₂ level was maintained with the double layer of Micropore Tape to support pollen respiration. The CO₂ level remained below levels toxic to the pollen during storage with the double layer of Micropore Tape, but the CO₂ was higher than that measured the single layer. Pollen tube germination assessments indicated a surface area of 71.3 mm² of Micropore Tape barrier in a single or double layer provides sufficient gas exchange to support pollen respiration while minimizing water vapor transmission from the storage container. An average seed set of 707 kernels/ear for the single layer and 663 kernels/ear for the double layer covering of Micropore Tape were observed, indicating the pollen was viable after 5 days of storage.

TABLE 36
Pollen viability assessment and Seed Set

| | | | Final | | |
|---|---|---|---|---|---|
| | | | PMC | | |
| | | | Relative | | |
| | | | to | | |
| | | Pollen Tube | Initial | | Average |
| Days | | Gemination | PMC | | Seed |
| Stored | Treatment | (%) | (%) | Replicates | Set (k) |
| 0 | Fresh | 58 | NA | NA | ND |
| | Pollen:Silica | | | | |
| | Mix | | | | |
| 5 | 1 layer of | 55 | 102.7 | 3 | 707 |
| | micropore | | | | |
| | tape | | | | |
| 5 | 2 layers of | 56 | 104.3 | 3 | 663 |
| | micropore | | | | |
| | tape | | | | |

NA = Not Applicable;
ND = No Data

### 6: Water Vapor Transmission of Tyvek, Micropore Tape, Open Perforation, and Parafilm

### Experiments 1 and 2: Water Vapor Transmission Rate Across a Tyvek Barrier, a 3M Micropore Tape Barrier, and an Open Perforation (No Covering) to Atmosphere

Two experiments were conducted to examine the water vapor transmission rate across a Tyvek and 3M micropore tape breathable barrier as compared to a perforation open to the atmosphere. All three breathable barriers had an identical surface area of 71.3 mm². The water vapor transmission rate was measured by weight loss of steady state water vapor flow over unit time over unit area of the breathable barriers under specific of temperature and humidity conditions. The measure of water vapor transmission rates was conducted at 23° C. and 58% to 61% relative humidity following similar approaches denoted in ASTM Standard Test Methods for Water Vapor Transmission of Materials (E96/E96M-16). See generally www.astm.org/standards/e96. An approximately 4-liter acrylic vacuum chamber with an internal dimensional size of 28 cm (11-inch)×28 cm (11-inch)×12.7 cm (5-inch) (Terra Universal model 5235-01B) was placed into a Fisher Scientific benchtop incubator (model 6500). A glycerol water solution was prepared at 80% weight of pure glycerol to deionized water. The specific gravity of the glycerol water solution was 1.4374. 500 ml of this glycerol water solution was placed into an 8.89 cm (3.5 inch) by 15.24 cm (6.0 inch) open container. The open container of glycerol water solution was placed into the acrylic chamber to provide the appropriate humidity within the chamber. Approximately 60 ml of deionized water was placed into each of twelve 125 ml Ball mason jars. The deionized water was approximately 19 mm (¾-inch) below the lid of each jar. Twelve mason jar lids were modified to include 71.3 mm² perforations using a handheld die press. Disks of Tyvek were cut from a standard FedEx envelope and was taped over the 3.175 mm perforations on three of the twelve lids. An impermeable plate seal film was applied to the outer perimeter of the Tyvek to seal the Tyvek to the container lid. 3M micropore tape was placed over the perforation on three of the twelve lids. Perforations on the remaining six of twelve lids were left uncovered. Lids were placed onto identical mason jars containing deionized water and tightly sealed using mason jar lid bands. All twelve sealed jars were inserted into the acrylic chamber and the acrylic chamber was placed into the Fisher incubator to maintain a controlled atmosphere for up to 8 days. Loss of weight measurements were collected to calculate the water vapor transmission rates from all twelve jars covering the three breathable barrier treatments.

The Tyvek breathable barrier was observed to reduce water vapor loss by 48.3% at 60% rH and 23° C. under static airflow compared to an open perforation. The micropore tape breathable barrier was observed to reduce water vapor loss by 23.2% at 60% rH and 23° C. under static airflow compared to an open perforation. The Tyvek barrier was more effective than the micropore tape barrier at reducing water vapor transmission for a storage vessel at 60% rH and 23° C. under static airflow by 32.6%.

TABLE 37
Water vapor loss through three different
71.3 mm² breathable barriers.

| | | | Water loss | Water loss |
|---|---|---|---|---|
| | Replicate | Water loss | (g/hour/ | (mol/hour/ |
| Treatment | Containers | (g/hour) | mm²) | mm²) |
| Open | 6 | 6.98 × 10⁻⁰³ | 9.80 × 10⁻⁰⁵ | 5.44 × 10⁻⁰⁶ |
| Perforation | | | | |
| Micropore | 3 | 5.36 × 10⁻⁰³ | 7.53 × 10⁻⁰⁵ | 4.18 × 10⁻⁰⁶ |
| Tape | | | | |
| Tyvek | 3 | 3.61 × 10⁻⁰³ | 5.07 × 10⁻⁰⁵ | 2.81 × 10⁻⁰⁶ |

### 7: Continuous Gas Analysis During Storage at 6° C.

### Experiment 1: Continuous Gas Usage During Pollen Storage with Breathable Barrier (9 Day)

In the continuous gas experiment, 9.699 g of pollen was mixed with 4.845 g of 10 μm crystalline silica (2:1 pollen:silica by weight) for a total of 14.53 g pollen mix with an initial fresh mix PMC of 36.39%. A total of 14.4 g pollen mix was added to a 400 mL Magenta GA7 box. A 50 ml conical tube cap containing 2.03 g soda lime was placed at the bottom of the GA7 box. The pollen mix was contained inside a PLA plastic cup (45 mm×30 mm) and the container was positioned above the open soda lime container. 100 ul of water was added to the bottom of the GA7 box due to the 30% rH in the laboratory environment during experiment setup. The addition of water was intended to increase the rate at which the internal atmosphere of the storage container reached a saturated relative humidity. An iButtonLink DS1923-F5 # Hygrochron temperature and humidity data logger was placed above the pollen mix container to monitor internal relative humidity of the pollen storage vessel. The GA7 lid was placed on top of the GA7 box and the gas exchange gap between box and lid was sealed by wrapping a single layer of 25.4 mm (1-inch) wide parafilm around the opening. The parafilm was stretched as is normal practice in plant tissue culture to create a tight seal. The GA7 gas exchange gap between box and lid surface area is calculated to total 42 mm². The pollen storage vessel was stored at 6° C. at 1 atm pressure for 9 days. A Quantek 902P gas analyzer was set up for continuous measurement of O₂ and CO₂. The input and output lines of the analyzer were attached to two 23-gauge precision glide needles and inserted into two septa installed in the side walls of the GA7. The input and output lines of the analyzer were run through the side wall of the refrigeration unit through a stopper. The analyzer was maintained outside of the refrigeration unit at room temperature. After 9 days, pollen tube germination, final PMC relative to initial PMC, and continuous gas analysis of O₂ and CO₂ was assessed.

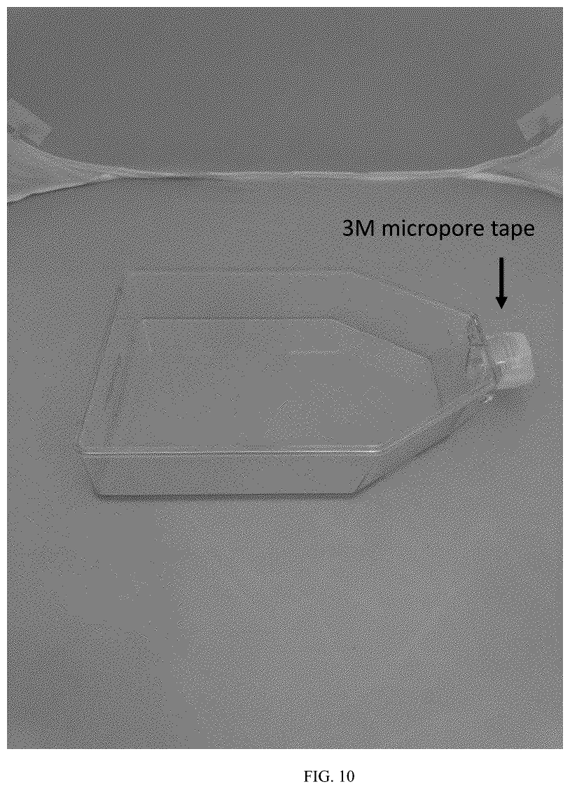

The iButtonLink DS1923-F5 # Hygrochron temperature and humidity data logger indicated a saturated rH above 98% was obtained throughout the duration of storage inside the storage vessel. Pollen tube germination was qualitatively scored a ranking of 2 (1%-20% germination rate; see FIG. 10) after 9 days. The relative PMC was 97.6%. The internal vessel rH and the final relative PMC data indicated that using parafilm as a breathable barrier to seal the 400 mL Magenta GA7 box reduced moisture loss from the storage vessel and pollen mix. Continuous internal gas sampling with a 1-minute sampling interval was acquired from the storage vessel during storage. Continuous gas data was normalized for atmospheric pressure changes. The O₂ content reduced to a minimum of 19.4% within the first few hours of storage at 6° C. and the CO₂ content remained below 0.1%. The internal O₂ content remained in steady state at 20.0% throughout the duration of the first 8 days of the storage experiment. On the 9th day, the O₂ content increased to 20.4%. This was likely due to a reduction in pollen viability and the associated decrease in pollen respiration. Use of parafilm as a breathable barrier provided sufficient gas exchange while minimizing water vapor transmission. The presence of soda lime provided sufficient sequestration of CO₂ in conjunction with the breathable barrier. The breathable barrier provided a high steady state level of oxygen to support pollen respiration. No reduction in oxygen content to anaerobic levels was measured inside the storage container.

8. Pollen Storage Scale Up Using a Non-Airtight Container with a Manufactured Aperture (i.e., 250 ml VWR® Cell Culture Flask) without Soda Lime

Experiments 1, 2, and 3: 1) Breathable barrier 250 ml cell culture flask using stock nitrocellulose barrier—without soda lime (5, 6, 7, 8, and 9 days); 2) Breathable barrier 250 ml cell culture flask stock cap vents covered with 3M Micropore Tape—without soda lime (5, 6, 7, 8, and 9 days); 3) Breathable barrier 250 ml cell culture flask with cap removed and entire opening covered with 1 layer 3M Micropore Tape—without soda lime (5, 6, 7, 8, and 9 days);

For experiments 1, 2, and 3, fresh pollen was collected and was mixed with 10 μm crystalline silica (2:1 pollen:silica by weight) on three different dates. The bulk pollen mixes were added directly to 250 ml VWR® cell culture flasks without added soda lime. Each experiment consisted of a different culture flask cap breathable barrier treatment. Each treatment for each experiment includes 1 replicate.

For experiment 1, a total of 51.15 g pollen mix (initial fresh mix PMC of 34.42%) was added to the 250 ml VWR® cell culture flask. The stock vented cap of the cell culture flask, with the stock imbedded nitrocellulose barrier, was placed onto the flask and tightened to create a seal. For experiment 2, a total of 48.4 g pollen mix (initial fresh mix PMC of 36.97%) was added directly to the 250 ml VWR® cell culture flask. The stock imbedded nitrocellulose barrier was removed from the vented cap and the vents were covered with one layer of 3M micropore tape. For experiment 3, a total of 18.06 g pollen mix (initial fresh mix PMC of 40.7%) was added directly to the 250 ml VWR® cell culture flask. The stock vented cap was removed entirely from the flask and the entire opening was covered with one layer 3M Micropore Tape. For experiments 1 and 2, the stock cap vents provided a total surface area of 36.4 mm² to act as breathable barrier. For experiment 3, removing the lid and covering the entire open neck of the flask with micropore tape created 486.95 mm² of breathable membrane surface area.

In each experiment, the pollen storage vessel was laid flat and stored at 6° C. at 1 atm pressure for 9 days. A subsample of pollen mix was acquired from each pollen storage vessel for pollinations onto one receptive ear on a hybrid maize plant on days 5, 6, 7, 8, and 9 of storage. On each day of sampling and pollinations, the pollen storage vessel was removed from storage, sampled, resealed with micropore tape in experiments 2 and 3, and placed back into storage at 6° C. In experiment 1, seed sets of 518 kernels/ear, 380 kernels/ear, 443 kernels/ear, 403 kernels/ear, and 467 kernels/ear were obtained at 5 days, 6 days, 7 days, 8 days, and 9 days, respectively. The 250 ml cell culture flask with the stock cap and nitrocellulose barrier provided sufficient gas exchange while maintaining pollen viability. In experiment 2, a seed set of 338 kernels/ear, 425 kernels/ear, 409 kernels/ear, and 2 kernels were obtained at 6 days, 7 days, 8 days, and 9 days, respectively. The stock cap without the imbedded nitrocellulose barrier and the vents covered with 3M Micropore Tape provided sufficient gas exchange to maintain pollen viability for up to 8 days. For experiment 3, a seed set of 457 kernels/ear, 517 kernels/ear, 461 kernels/ear, 30 kernels/ear, and 27 kernels/ear were obtained at 5 days, 6 days, 7 days, 8 days, and 9 days, respectively. The 3M micropore tape covering the 250 ml cell culture flask open neck provided sufficient gas exchange while maintaining pollen viability for up to 9 days. Results of seed set in each experiment indicates that the presence of soda lime was not required to maintain CO₂ below levels toxic and that the use of a breathable barrier provided sufficient gas exchange to support aerobic respiration. A cell culture flask is a suitable pollen storage vessel, and the open neck of the flask neck provides sufficient surface area of gas exchange through a breathable barrier while minimizing water vapor transmission from the vessel.

TABLE 38
Seed set results from the pollen scale up using
250 ml VWR ® cell culture flasks

| | | | Breathable | |
|---|---|---|---|---|
| | | | Barrier | |
| | | | Surface | |
| | Days | | Area | Seed |
| Experiment | Stored | Breathable Barrier | (mm²) | Set(k) |
| 1 | 5 | Nitrocellulose | 36.4 | 518 |
| 1 | 6 | Nitrocellulose | 36.4 | 380 |
| 1 | 7 | Nitrocellulose | 36.4 | 443 |
| 1 | 8 | Nitrocellulose | 36.4 | 403 |
| 1 | 9 | Nitrocellulose | 36.4 | 467 |
| 2 | 5 | Micropore Tape | 36.4 | ND |
| 2 | 6 | Micropore Tape | 36.4 | 338 |
| 2 | 7 | Micropore Tape | 36.4 | 425 |
| 2 | 8 | Micropore Tape | 36.4 | 409 |
| 2 | 9 | Micropore Tape | 36.4 | 2 |
| 3 | 5 | Micropore Tape | 486.95 | 457 |
| 3 | 6 | Micropore Tape | 486.95 | 517 |
| 3 | 7 | Micropore Tape | 486.95 | 461 |
| 3 | 8 | Micropore Tape | 486.95 | 30 |
| 3 | 9 | Micropore Tape | 486.95 | 27 |

ND = No Data

9. Breathable Barrier Pollen Storage with Crystalline Silica, Metallic Powders, and Mica Carriers

Crystalline quartz silica is an effective carrier for maize pollen storage. Crystalline silica inhibits clumping interaction between pollen grains during storage but does not excessively coat pollen grains immediately upon application or due to carrier particle breakup during handling. In addition, crystalline silica does not act as a desiccant in storage.

Metallic powders are effective carriers for pollen storage. Metallic powders prevent clumping interaction between adjacent pollen grain barriers during storage, but do not excessively coat the pollen barrier surface and do not inhibit adherence to maize silks or other plant stigmas. This lack of inhibition enables effective pollen tube germination. Elemental metallic powders, metallic oxide powders, and metallic carbide powders are all effective pollen storage carriers. These powders may be manufactured by diverse techniques to optimize function, including solid-state reduction, electrolysis, chemical reactions, high-temperature combustion, gas atomization, ultra-high pressure water atomization, pressing and sintering, centrifugal atomization, grinding, and other polishing techniques to optimize particle size and particle surface properties. The optimal particle type for metallic powders in maize pollen storage is believed to be 10 μm polished spherical particles, but other particle sizes and surface characteristics may be superior for other pollen types. In some applications, metallic powders may be coated in polymers to modify particle surface interaction with pollen membranes. In other applications, metallic particles may be coated in active ingredients to modify interaction with pollen grain membranes, modify the respiration of pollen and microbes in storage, or inhibit microbial proliferation during storage. These active ingredients may include nucleic acids, proteins, pesticides, or bio-stimulants. Metallic carriers include elements with known biological roles in plants that may enhance pollen performance and those with no known biological role that have no impact on pollen performance. Ferro-magnetic carriers may be preferred in applications where the carrier can be magnetically removed from the pollen carrier mix following storage to enrich the concentration of pollen in the mix.

Micas are a group of minerals defined by a general chemical formula and perfect basal cleavage. Perfect basal cleavage results in flat sheet shaped particles that are effective in preventing interaction between adjacent pollen grain membranes. In addition to physical properties that make mica minerals effective carriers in pollen storage, the high reflectivity of mica minerals can act as a visual indicator during pollen application. These reflective properties can be visualized by protocol operators or machine cameras to track the distribution of pollen during application or verify where pollinations have taken place.

Table 39 details the performance of crystalline silica, metallic powder carriers, and mica as a carrier in pollen storage. All carriers in this test show similar performance to crystalline silica detailed in U.S. Provisional Application No. 63/289,299, filed Dec. 14, 2021, incorporated herein by reference.

TABLE 39
Seed set from pollen stored with ten carriers.

| | Days | Avg Seed | |
|---|---|---|---|
| Carrier | Stored | Set/Ear (k) | Stdev (k) |
| 10 μm Crystalline Silica | 5 | 401 | 102 |
| 10 μm Mica | 5 | 276 | 160 |
| 10 μm Silicon Carbide | 5 | 399 | 92 |
| 10 μm 316 L Stainless Steel Powder | 5 | 362 | 92 |
| 10 μm 316 L Stainless Steel Powder | 5 | 497 | 157 |
| Optimized for 3D Printing | | | |
| 10 μm Iron Powder | 5 | 505 | 57 |
| 10 μm Spherical Aluminum | 5 | 440 | 54 |
| 5 μm Spherical Aluminum Oxide | 5 | 415 | 125 |
| 10 μm Titanium Powder | 5 | 686 | 148 |
| 10 μm Chromium Powder | 5 | 535 | 77 |

All carriers were mixed with pollen at a ratio of two parts pollen, one part carrier by weight. The 10 μm 316L stainless steel powder is produced through high-temperature combustion and individual particles have an amorphous structure. It is optimized for 3D printing through ultra-high-pressure water and gas atomization with grinding to produce uniform, spherical particles.

### 10. Pollen Storage of Plants Containing Transgenic Events

Male ratings are an overall assessment of performance as a pollen source that accounts for all data types collected during inbred parent line development. Ratings from highest to lowest male performance are Desirable, Acceptable, Marginal, and Do Not Advance.

Pollen for five inbred parent lines containing transgenic events was collected, separately, and mixed with a crystalline silica carrier at a ratio of two parts pollen, one part carrier by weight. A portion of the pollen mixed with crystalline silica was used to conduct fresh self-pollinations (stored zero days). The remaining pollen plus crystalline silica mix was stored in a sealed vessel with added soda lime in a 6° C. environment. After five days, stored pollen was applied to silks on the same inbred line that provided the pollen (i.e., a self-pollination). All ears received the same quantity of fresh pollen and carrier or stored pollen and carrier by volume.

TABLE 40
Seed set from stored pollen for five different inbred
parent lines. Inbred lines one through four comprise
transgenic events Bt11, GA21, and MIR162. Inbred line
five comprises transgenic events Bt11 and MIR162.

| | | Days | Avg Seed | |
|---|---|---|---|---|
| Tester Line | Male Rating | Stored | Set/Ear (k) | Stdev (k) |
| Inbred Line 1 | Marginal | 0 | 301 | 47 |
| | | 5 | 44 | 38 |
| Inbred Line 2 | Acceptable | 0 | 53 | 22 |
| | | 5 | 173 | 133 |
| Inbred Line 3 | Marginal | 0 | 319 | 181 |
| | | 5 | 242 | 134 |
| Inbred Line 4 | Acceptable | 0 | 173 | 136 |
| | | 5 | 3 | 5 |
| Inbred Line 5 | Do Not Advance | 0 | 59 | 43 |
| | | 5 | 31 | 53 |

### 11. Breathable Barrier Pollen Storage—Vessel Design

### Experiment 1: A 25 ml VWR® Cell Culture Flask with Vented Cap Compared to an 850 ml VWR® Cell Culture Flask with Pollen Stored from the Same Collection

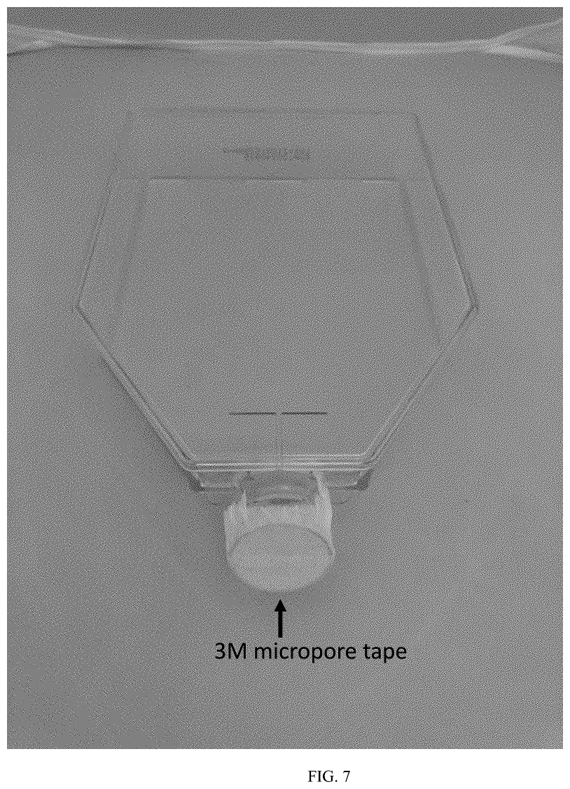

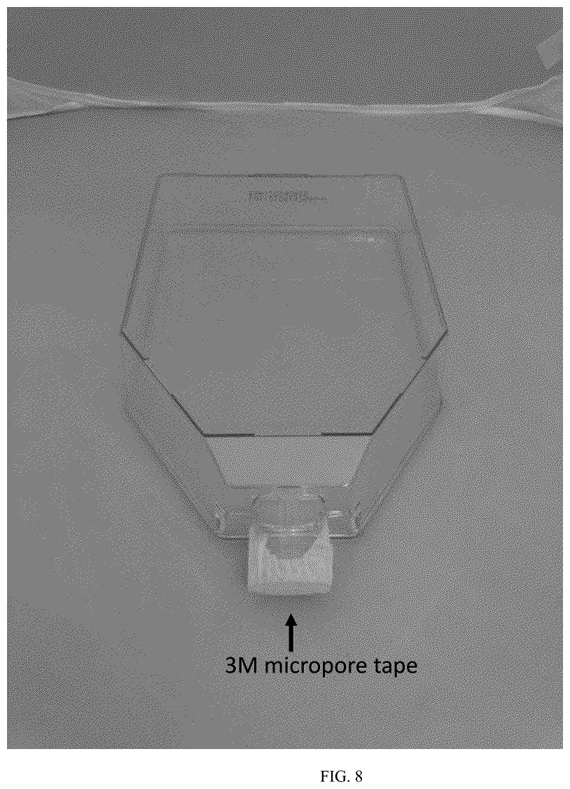

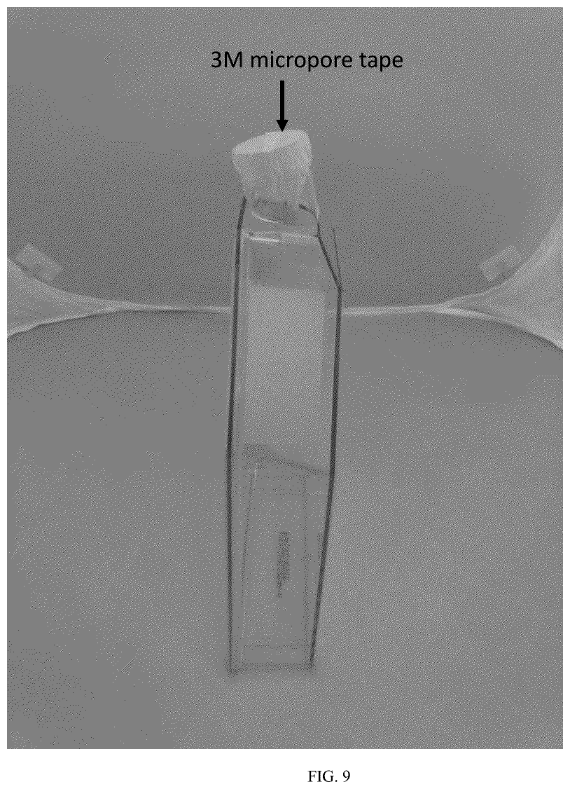

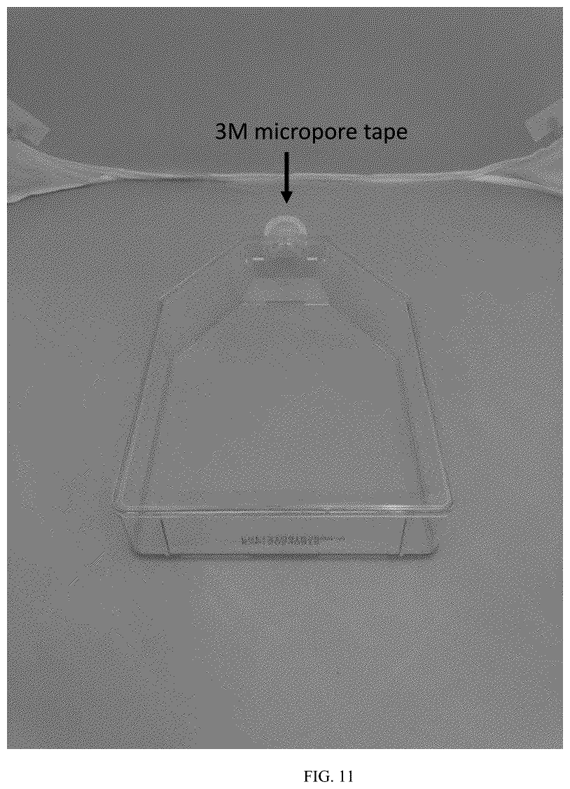

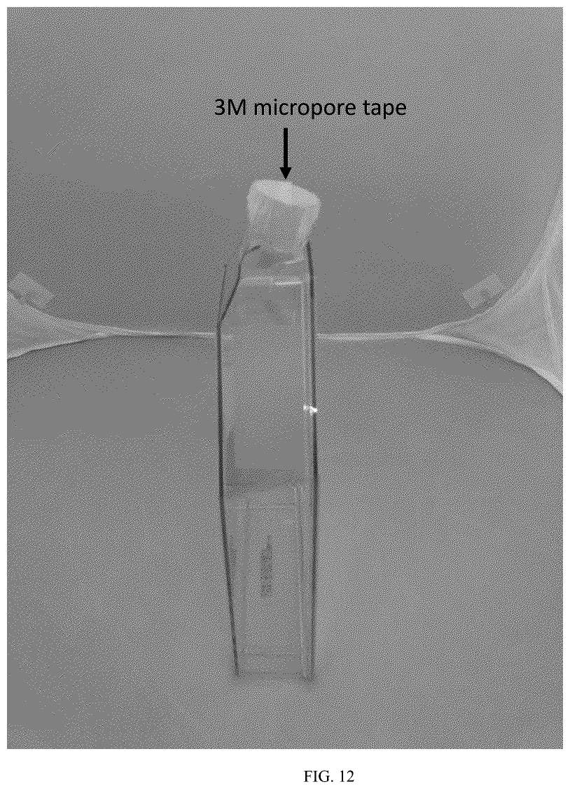

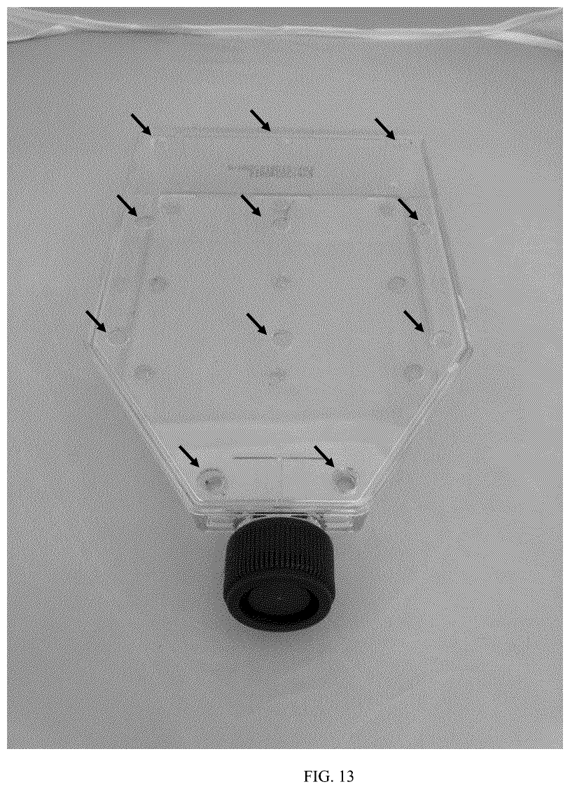

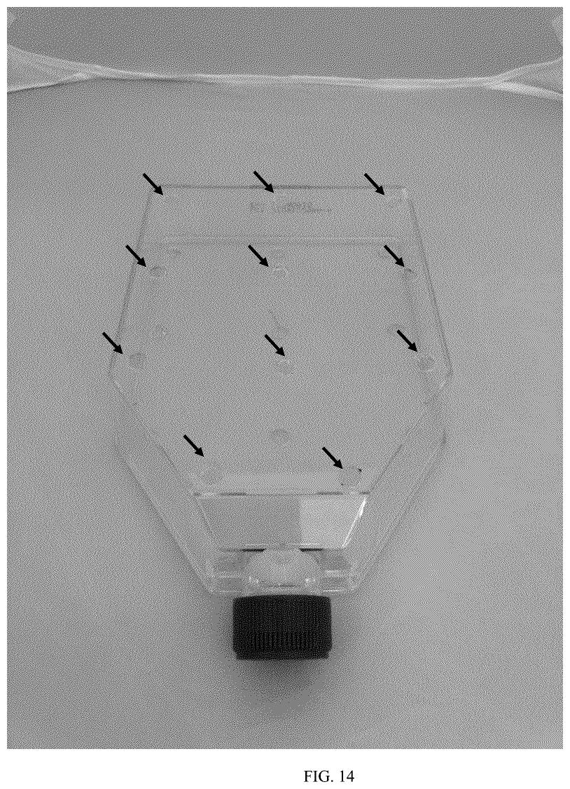

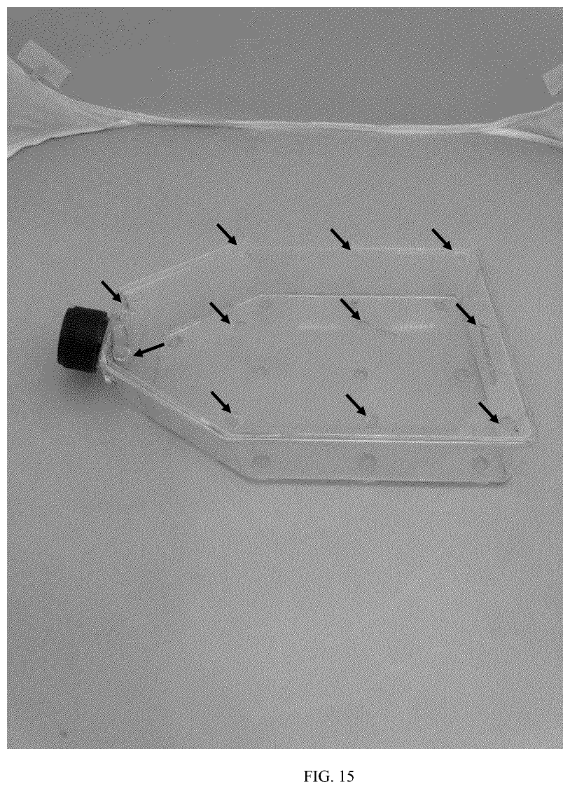

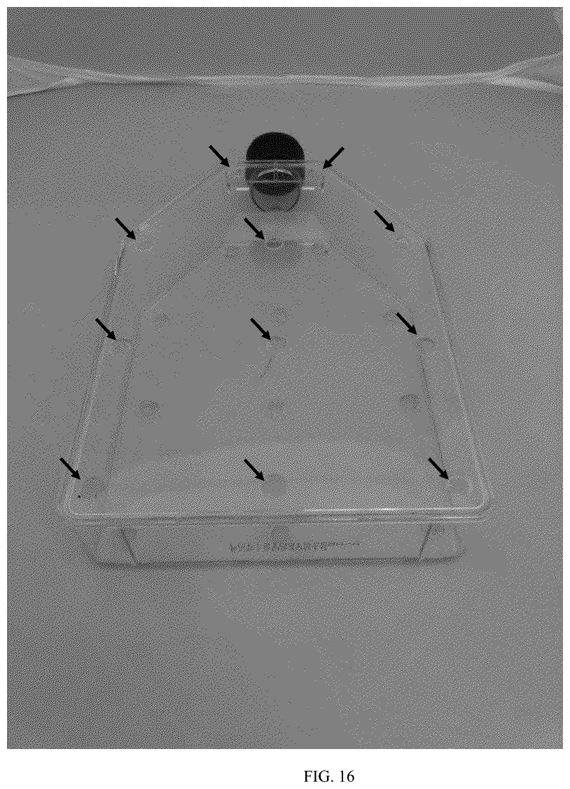

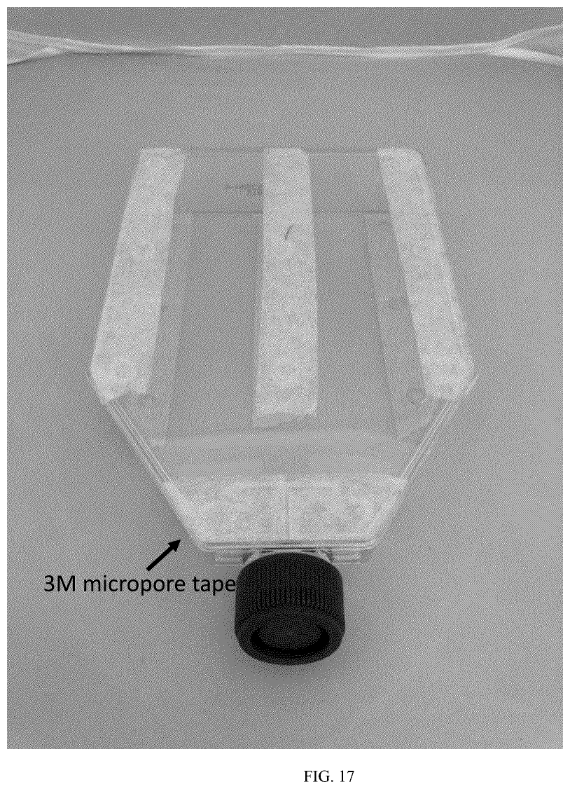

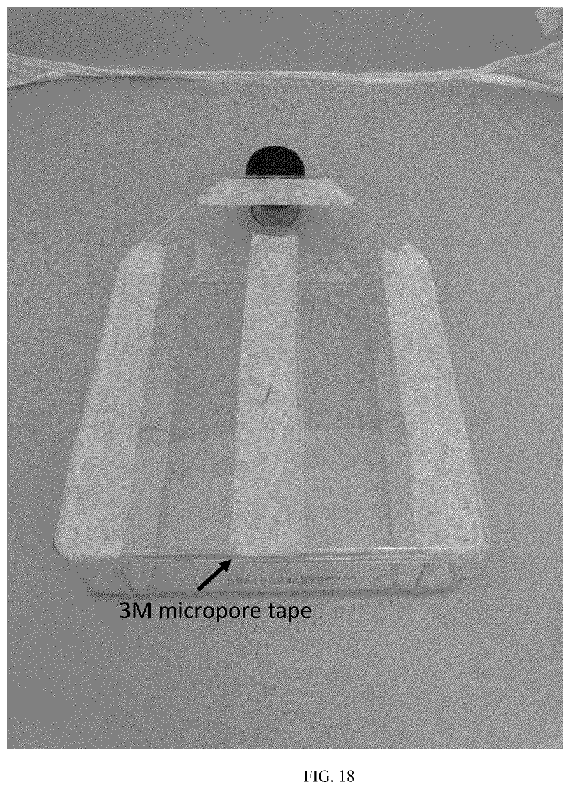

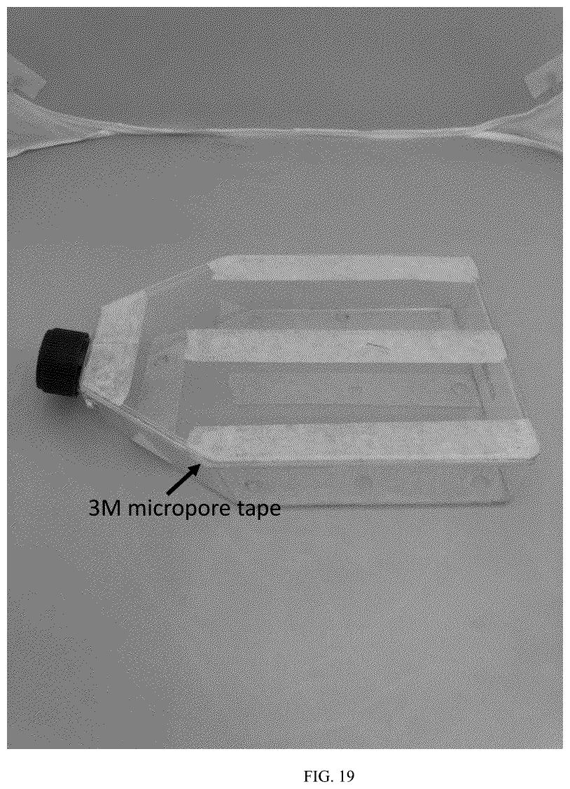

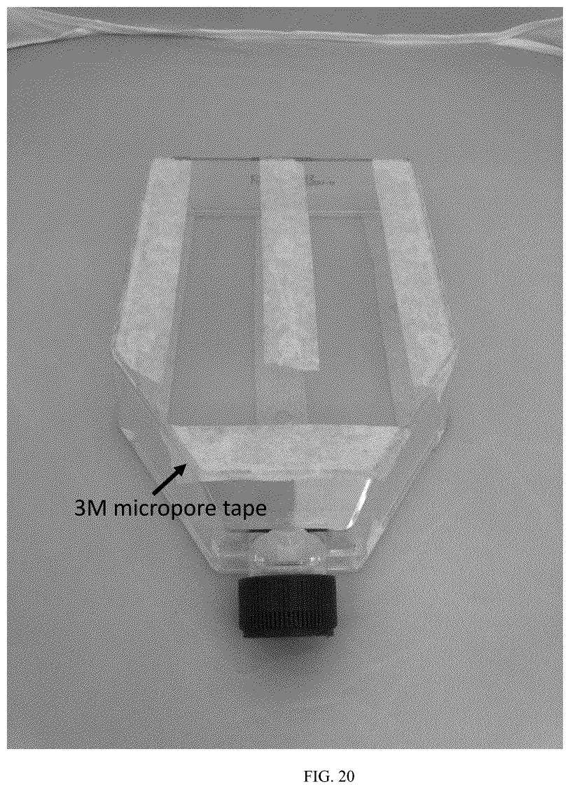

Pollen was collected from a field trial and mixed with the silica carrier at a ratio of two parts pollen to one part silica by weight. The total amount of pollen collected after mixing with silica was 1,009 grams. One-thousand grams of the treated pollen was placed into the 850 ml VWR® Cell Culture Flask, the flask cap was removed, and the vessel mouth was covered with a single layer of 3M micropore tape to act as a breathable barrier (see, for example, FIGS. 7-12). This storage vessel configuration provided 660.5 mm² total breathable barrier surface area at a rate of 0.7 mm² of surface area per gram of treated pollen. In this configuration, the maximum distance between a pollen grain and the breathable barrier was calculated to be 274 mm based on trigonometry. Nine grams of the treated pollen was placed into a 25 ml VWR® Cell Culture Flask and the stock vented cap with a nitrocellulose membrane was used as a breathable barrier. This storage vessel configuration provided 15.2 mm² total breathable barrier surface area at a rate of 1.7 mm² of surface area per gram of treated pollen. In this configuration, the maximum distance between a pollen grain and the breathable barrier was measured at 72 mm. Oxygen content of air within the pore spaces between pollen grains and silica carrier particles within the 850 ml flask was measured during storage by inserting a needle through the breathable barrier or through a gas tight septum installed in the rear of the storage vessel. Oxygen content of the air within the pore space was found to have fallen as low as 0.7% within the first two hours of storage and was observed to stabilize at 13.1% over the duration of five days of storage at 6° C. After five days of storage, pollen from each storage vessel was separately mixed to homogenously combine pollen stored throughout each storage vessel. The mixed pollen was applied to pollen tube germination plates and tester ears. All ears received the same quantity of stored pollen.

TABLE 41
Seed set obtained from pollen stored for five days comparing an unmodified 850
ml VWR ® Cell Culture Flask to an unmodified 25 ml VWR ® Cell Culture Flask.

| | | | | | Maximum | | | |
|---|---|---|---|---|---|---|---|---|
| | | | | | Distance | | | |
| | | | | Breathable | Between | | | |
| | | | | Membrane | Stored | | | |
| | | Total | | Surface | Pollen and | | | |
| | | Breathable | Stored | Area per | the Nearest | Pollen | | |
| | Interior | Membrane | Treated | gram of | Breathable | Tube | Avg Seed | |
| Storage | Volume | Surface Area | Pollen | pollen | Barrier | Gemination | Set/Ear | Stdev |
| Vessel | (ml) | (mm²) | Mass (g) | (mm²) | (mm) | (%) | (k) | (k) |
| 25 ml | 35 | 15.2 | 9 | 1.7 | 72 | 55 | 451 | 131 |
| Flask | | | | | | | | |
| Unmodified | 1350 | 660.5 | 1000 | 0.7 | 274 | 47 | 328 | 132 |
| 850 ml | | | | | | | | |
| Flask | | | | | | | | |

### Experiment 2. Modified 850 ml VWR® Cell Culture Flask

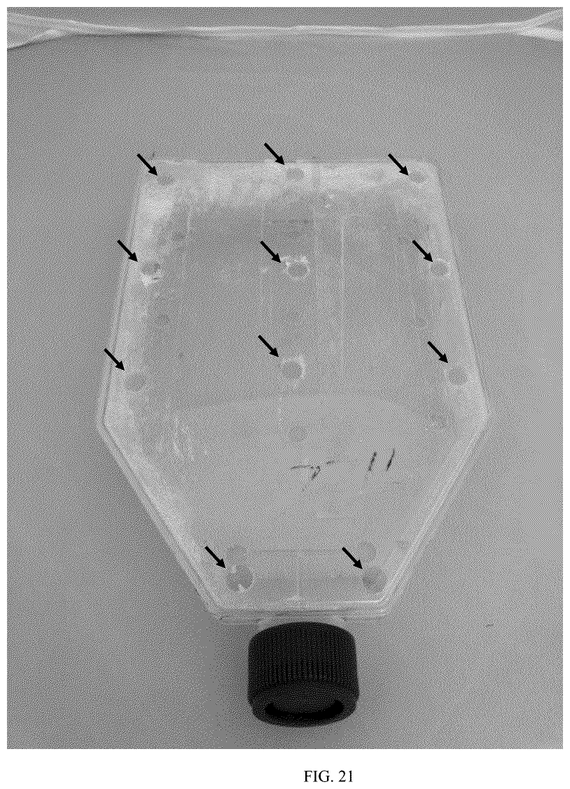

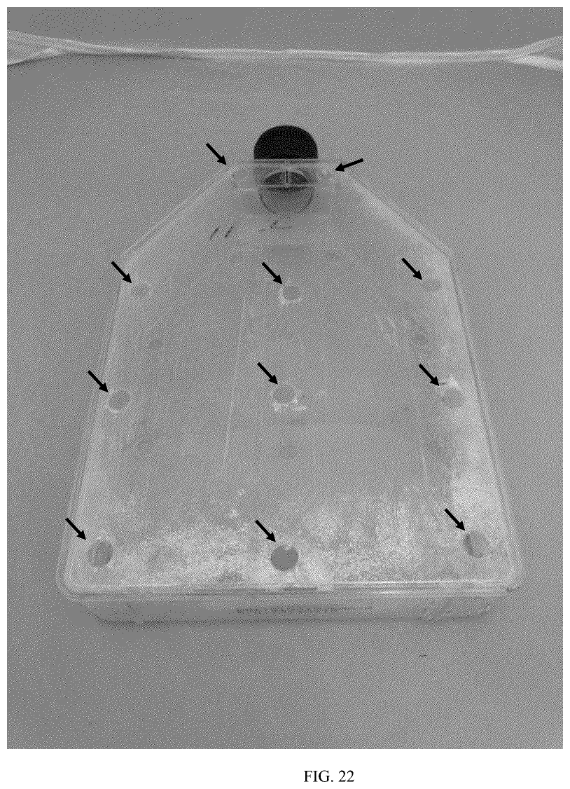

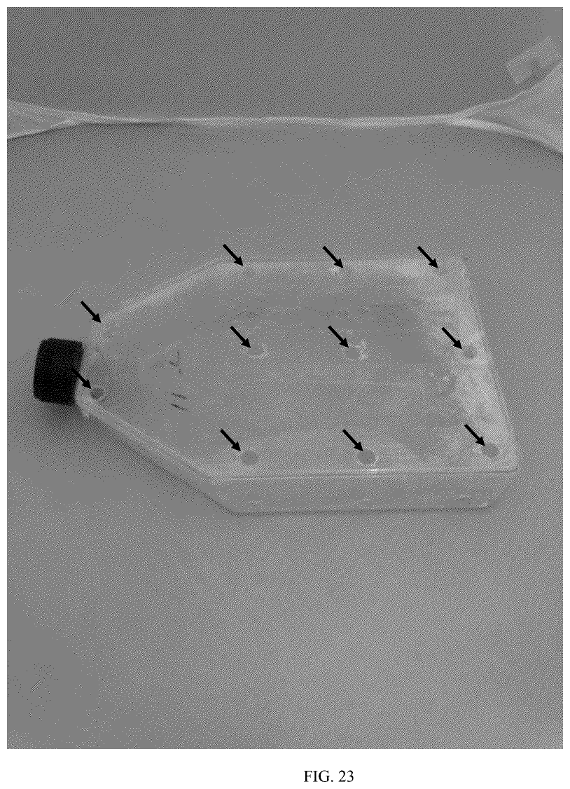

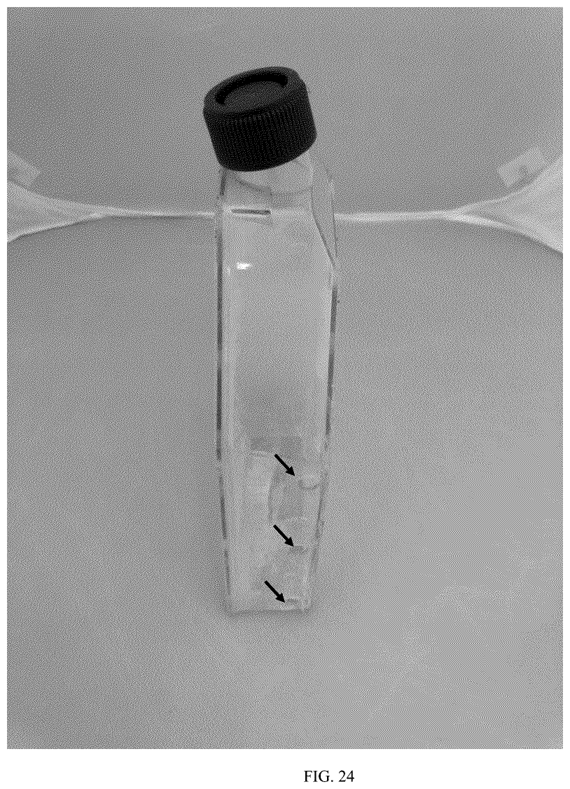

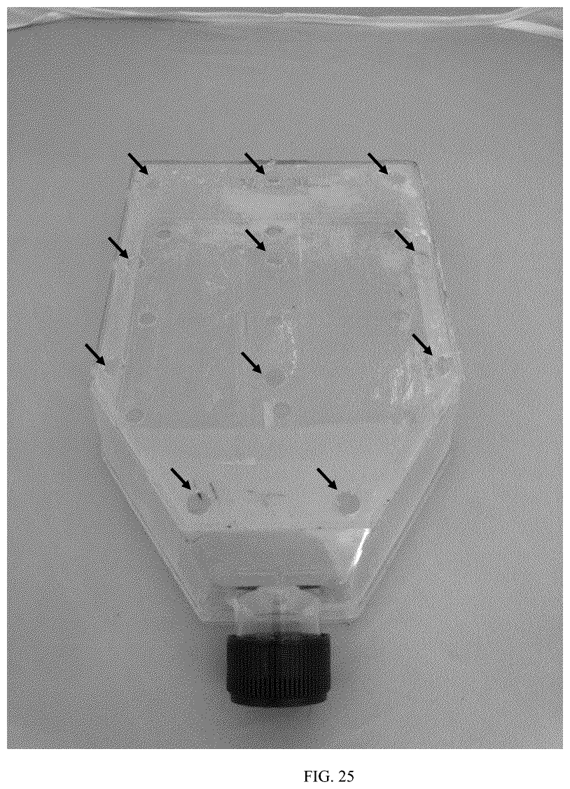

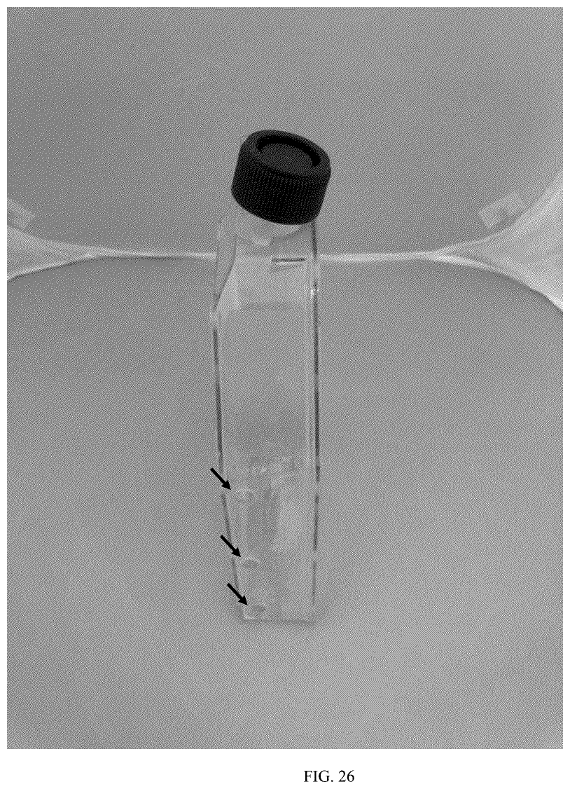

After observing the lagging seed set performance of pollen stored in an 850 ml flask when compared to the same batch of pollen stored in a 25 ml flask, it was hypothesized that the scaled-up storage conditions may have impacted pollen vigor. Anaerobic conditions early in the storage process and reduced oxygen availability throughout the duration of storage was hypothesized to have reduced pollen vigor such that pollen, which tested as viable on pollen tube germination media, was not able to complete pollen tube growth and fertilization before dying of environmental stress when placed on a silk. To test this hypothesis, an 850 ml flask was modified to reduce the maximum distance between pollen grains and the nearest breathable barrier while still maintaining the total breathable barrier surface area to grams of pollen ratio within established metrics. The flask was modified by drilling 28 holes 10 mm in diameter at selected points on the flask. Nine holes were drilled through the larger surface area flat side of the flask in a three-by-three grid pattern with 72 mm spacing between holes. Hole centers were 12 mm from the nearest edge where applicable. This larger surface area flat side is designed to be the upward facing top of the flask during use. The same three by three grid pattern of 10 mm holes was drilled through the smaller surface area flat side of the flask with 64 mm spacing between holes. Hole centers were 12 mm from the nearest edge where applicable. This smaller surface area flat side is designed to the downward facing bottom of the flask during use and is lower in surface area due to a taper manufactured into the 47 mm exterior height sidewalls of the flask. Two additional 10 mm holes were drilled into the larger surface area top side of the flask at the two front-facing obtuse angled corners. Each hole center was 12 mm from the two nearest edges of the flask. Two additional 10 mm holes were drilled into the smaller surface area bottom side of the flask at the two front-facing obtuse angled corners. Each hole center was 12 mm from the two nearest edges of the flask. Three 10 mm holes were drilled into each side of the flask on the axis running parallel to the direction of the vessel mouth for a total of six additional holes. These holes were placed with 64 mm spacing, and each hole center was 12 mm from the bottom facing edge of the flask. See, for example, FIGS. 21-26.

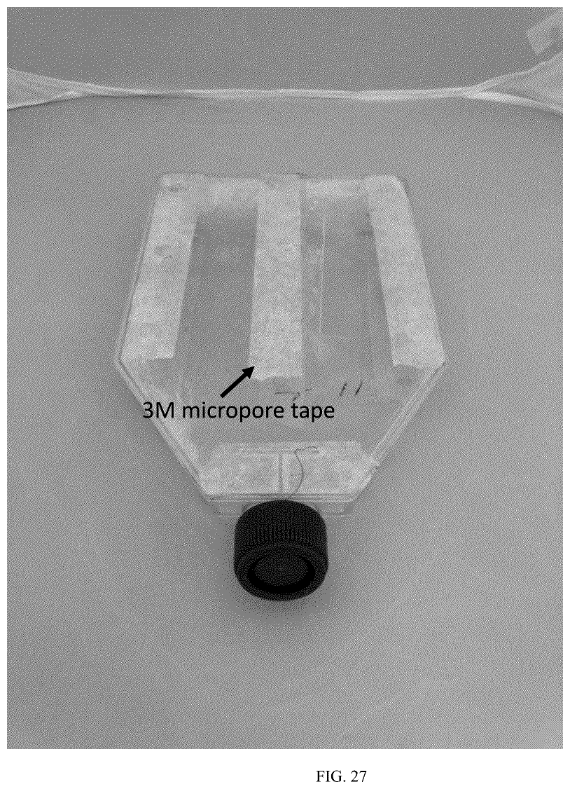

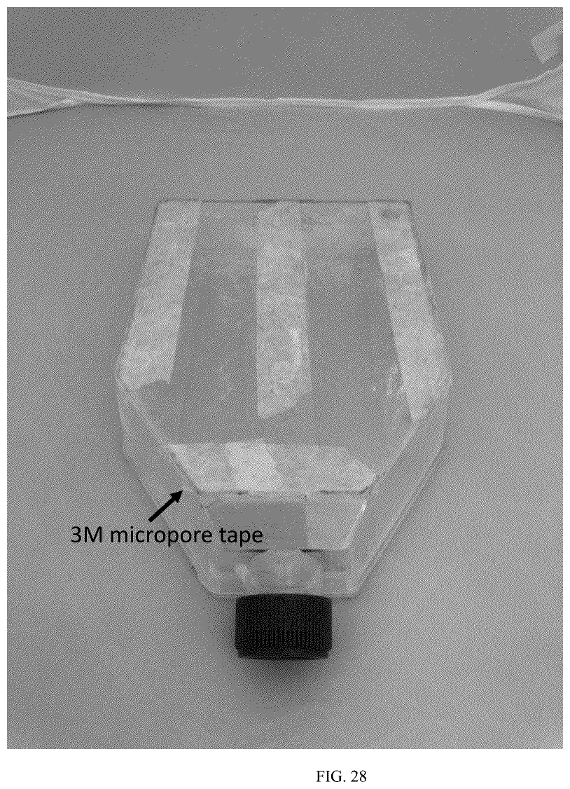

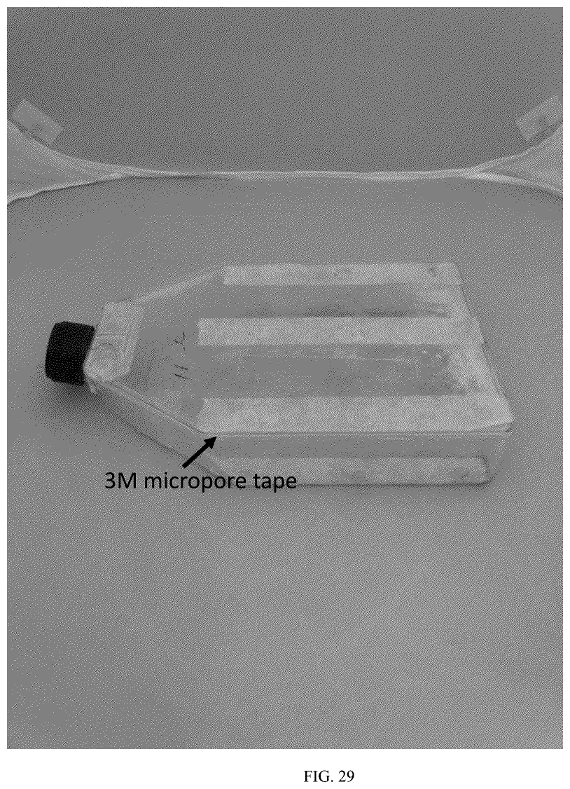

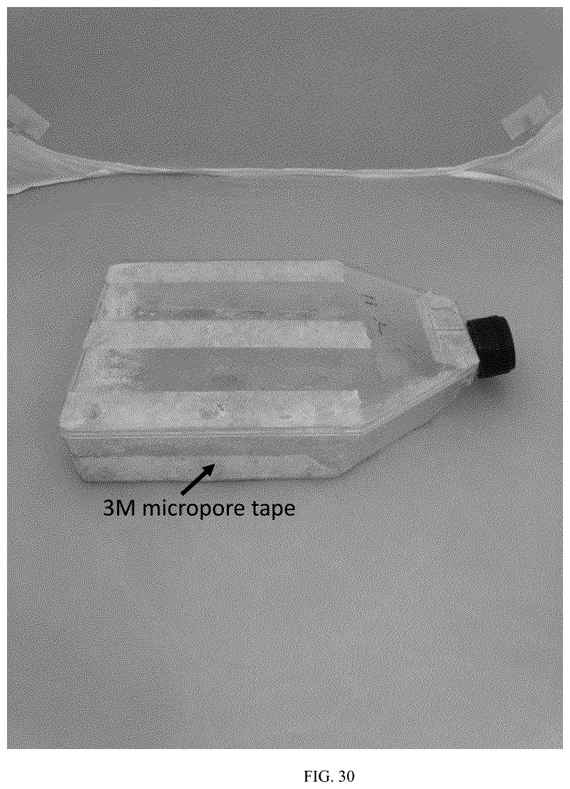

For experiment 2, pollen was collected from a field trial and mixed with the silica carrier at a ratio of two parts pollen to one part silica by weight. The total amount of pollen collected after mixing with silica was 860 grams. Eight hundred fifty grams of the treated pollen was placed into the optimized 850 ml flask with 28 added 10 mm holes. The non-vented vessel cap was tightly sealed on the vessel mouth and each of the 28 added 10 mm holes was covered in a single layer of 3M micropore tape (see, for example, FIGS. 27-30). This storage vessel configuration provided 2199.1 mm² total breathable barrier surface area at a rate of 2.6 mm² of surface area per gram of treated pollen. In this configuration, the maximum distance between a pollen grain and the breathable barrier was calculated to be 47 mm based on trigonometry. Ten grams of the treated pollen was placed into the 25 ml flask, and the stock vented lid with a nitrocellulose membrane was used as a breathable barrier. This storage vessel configuration provided 15.2 mm² total breathable barrier surface area at a rate of 1.5 mm² of surface area per gram of treated pollen. In this configuration, the maximum distance between a pollen grain and the breathable barrier was measured at 72 mm. Oxygen content of air within the pore spaces between pollen grains within the optimized 850 ml flask was measured during storage by inserting a needle through a breathable barrier or through a gas tight septum installed in the rear of the storage vessel. Oxygen content of the air within the pore space did not fall below 18.7% over six days of storage. After six days of storage, pollen from each storage vessel was separately mixed to homogenously combine pollen stored throughout each storage vessel. The mixed pollen was applied to pollen tube germination plates and tester ears. All ears received the same quantity of stored pollen.

TABLE 42
Seed set obtained from pollen stored for six days comparing the optimized 850
ml VWR ® Cell Culture Flask to an unmodified 25 ml VWR ® Cell Culture Flask.

| | | | | | Maximum | | | |
|---|---|---|---|---|---|---|---|---|
| | | | | | Distance | | | |
| | | | | Breathable | Between | | | |
| | | | | Membrane | Stored | | | |
| | Storage | Total | Stored | Surface | Pollen and | | | |
| | Vessel | Breathable | Treated | Area per | the Nearest | Pollen | | |
| | Interior | Membrane | Pollen | gram of | Breathable | Tube | Avg Seed | |
| Storage | Volume | Surface Area | Mass | pollen | Barrier | Gemination | Set/Ear | Stdev |
| Vessel | (ml) | (mm²) | (g) | (mm²) | (mm) | (%) | (k) | (k) |
| 25 ml | 35 | 15.2 | 10 | 1.5 | 72 | 73 | 308 | 141 |
| Flask | | | | | | | | |
| Optimized | 1350 | 2199.1 | 850 | 2.6 | 47 | 63 | 421 | 180 |
| 850 ml | | | | | | | | |
| Flask | | | | | | | | |

### Experiment 3. An Unmodified 850 ml VWR® Cell Culture Flask Compared to an Optimized 850 ml VWR® Cell Culture Flask

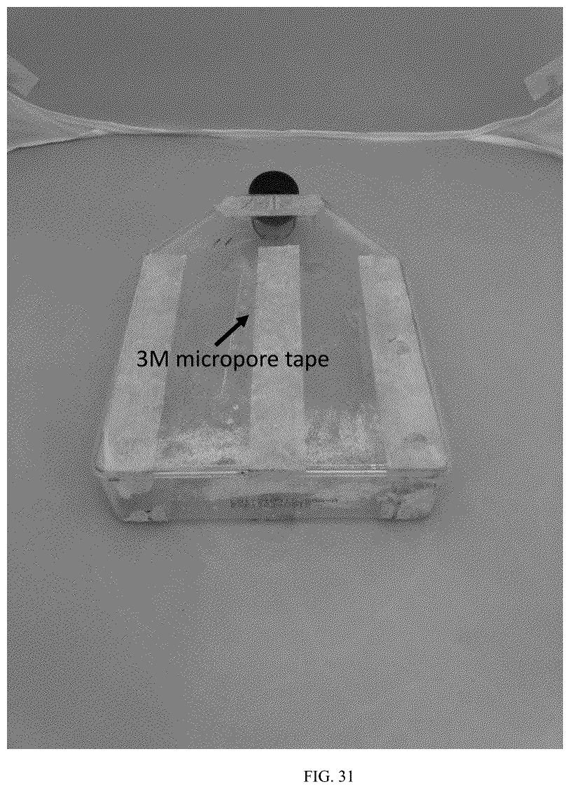

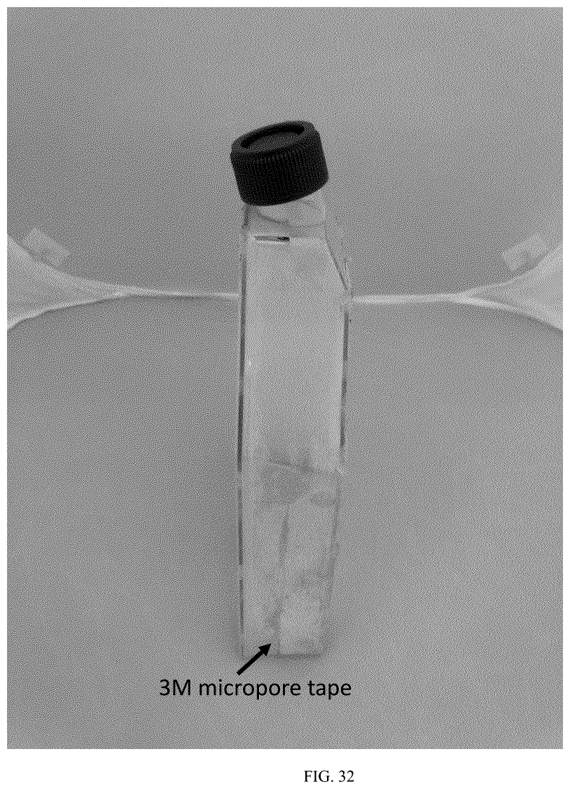

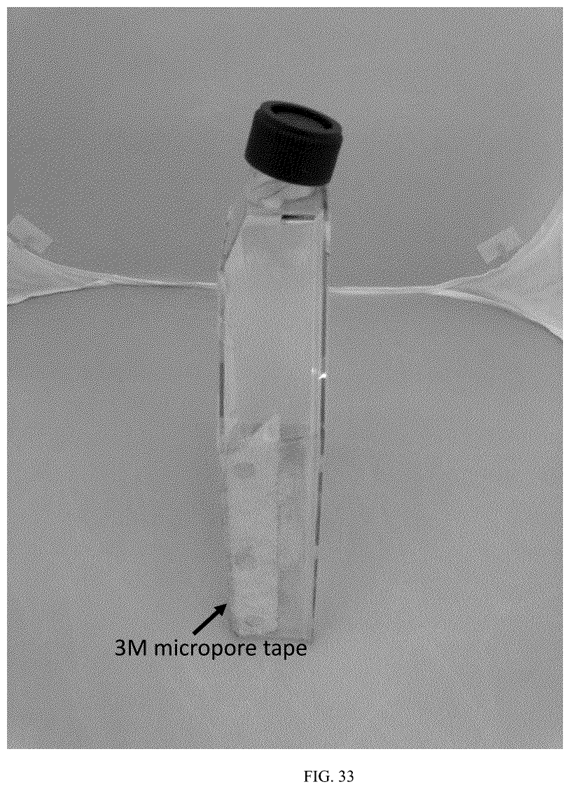

After observing the increased seed set obtained from pollen stored in an optimized cell culture flask, an experiment was conducted to directly compare the performance of equivalent pollen stored in an unmodified 850 ml flask and an optimized 850 ml flask with 28 added openings for breathable barriers (see, for example, FIGS. 21-26). The same optimized 850 ml flask with 28 added openings for breathable barriers described in Experiment 2 was used during Experiment 3. Pollen was collected from a field trial and mixed with the silica carrier at a ratio of two parts pollen to one part silica by weight. The total amount of pollen collected after mixing with silica was 2,000 grams. One thousand grams of the treated pollen was placed into the optimized 850 ml flask with 28 added 10 mm holes. The non-vented vessel cap was left tightly sealed on the vessel mouth and each of the 28 added 10 mm holes was covered in a single layer of 3M micropore tape (see, for example, FIGS. 27-31). This storage vessel configuration provided 2199.1 mm² total breathable barrier surface area at a rate of 2.2 mm² of surface area per gram of treated pollen. In this configuration, the maximum distance between a pollen grain and the breathable barrier calculated to be 47 mm based on trigonometry. One thousand grams of the treated pollen was placed into an unmodified 850 ml flask, the flask cap removed, and the vessel mouth was covered with a single layer of 3M micropore tape to act as a breathable barrier. This storage vessel configuration provided 660.5 mm² total breathable barrier surface area at a rate of 0.7 mm² of surface area per gram of treated pollen. In this configuration, the maximum distance between a pollen grain and the breathable barrier was calculated to be 274 mm based on trigonometry. After three days of storage, pollen from each storage vessel was separately mixed to homogenously combine pollen stored throughout each storage vessel. The mixed pollen was applied to pollen tube germination plates and tester ears. All ears received the same quantity of stored pollen. The mixed pollen was also mechanically broadcast over receptive ears to further test pollination efficiency. Pollen dosage to individual ears was not equivalent during broadcast application.

TABLE 43
Seed set obtained from pollen stored for three days comparing an optimized 850
ml VWR ® Cell Culture Flask to an unmodified 850 ml VWR ® Cell Culture Flask.

| | | | | | Maximum | | |
|---|---|---|---|---|---|---|---|
| | | | | | Distance | | |
| | | | | Breathable | Between | | |
| | | | | Membrane | Stored | | Mechanized |
| | Storage | Total | | Surface | Pollen and | Manual | Broadcast |
| | Vessel | Breathable | Stored | Area per | the Nearest | Pollination | Pollination |
| | Interior | Membrane | Treated | gram of | Breathable | Avg Seed | Avg Seed |
| Storage | Volume | Surface Area | Pollen | pollen | Barrier | Set/Ear | Set/Ear |
| Vessel | (ml) | (mm²) | Mass (g) | (mm²) | (mm) | (k) | (k) |
| Unmodified | 1350 | 660.5 | 1000 | 0.7 | 274 | 61 | 3 |
| 850 ml | | | | | | | |
| Flask | | | | | | | |
| Optimized | 1350 | 2199.1 | 1000 | 2.2 | 47 | 148 | 41 |
| 850 ml | | | | | | | |
| Flask | | | | | | | |

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. An apparatus to store pollen, comprising a vessel with a total breathable barrier surface area.

42. The apparatus of claim 41, wherein the vessel comprises multiple openings.

43. The apparatus of claim 41, wherein the vessel comprises a total breathable barrier surface area between 0.49 mm² per gram of fresh or treated pollen and 47.5 mm² per gram of fresh or treated pollen.

44. The apparatus of claim 42, wherein the vessel comprises at least one opening, at least two openings, at least three openings, at least four openings, at least five openings, at least six openings, at least seven opening, at least eight openings, at least nine openings, at least ten openings, at least eleven openings, at least twelve openings, at least thirteen openings, at least fourteen openings, at least fifteen openings, at least sixteen openings, at least seventeen openings, at least eighteen openings, at least nineteen openings, at least twenty openings, at least twenty-one openings, at least twenty-two openings, at least twenty-three openings, at least twenty-four openings, at least twenty-five openings, at least twenty-six openings, at least twenty-seven openings, at least twenty-eight openings, at least twenty-nine openings, or at least thirty openings.

45. The apparatus of claim 44, wherein the at least one opening has a total surface area of 15.2 mm² to 660.5 mm².

46. (canceled)

47. (canceled)

48. (canceled)

49. (canceled)

50. (canceled)

51. (canceled)

52. The apparatus of claim 42, wherein the multiple openings comprise a total combined surface area ranging between 0.49 mm² per gram of fresh or treated pollen and 47.5 mm² per gram of fresh or treated pollen.

53. (canceled)

54. (canceled)

55. (canceled)

56. (canceled)

57. The apparatus of claim 42, wherein the multiple openings are individually covered in a breathable barrier, wherein the multiple openings are distributed such that no pollen grain is greater than 47 mm from the nearest breathable barrier, or any other distribution that optimizes the farthest possible distance between pollen grains and the nearest breathable barrier.

58. The apparatus of claim 57, wherein the breathable barrier is selected from the group consisting of Parafilm, Tyvek, 3M Micropore tape, cellulose, nitrocellulose, and a non-airtight container.

59. The apparatus of claim 58, wherein the breathable barrier is 3M Micropore tape.

60. (canceled)

61. (canceled)

62. (canceled)

63. (canceled)

64. The apparatus of claim 41, wherein the vessel is any brand of cell culture flask in a stock or modified configuration.

65. The apparatus of claim 41, wherein the vessel is any plastic, metal, or ceramic container with one or more openings for breathable barriers.
